(19) United States
(12) Patent Application Publication
(10) Pub. No.: US 2018/0188139 A1
(43) Pub. Date: Jul. 5, 2018

(54) IMPROVED ASSAYS AND METHODS FOR ALLERGEN DETECTION

(71) Applicant: DOTS Technology Corp., Natick, MA (US)

(72) Inventors: Adi Gilboa-Geffen, Wayland, MA (US); Renuka Babu Brown, Weston, MA (US)

(21) Appl. No.: 15/736,801

(22) PCT Filed: Jun. 20, 2016
(86) PCT No.: PCT/US16/38412

§ 371 Date: Dec. 15, 2017

Related U.S. Application Data
(60) Provisional application No. 62/305,134, filed on Mar. 8, 2016.
(60) Provisional application No. 62/187,871, filed on Jul. 2, 2015.
(60) Provisional application No. 62/182,718, filed on Jun. 22, 2015.

(51) Int. Cl. G01N 1/28; G01N 33/543; G01N 33/53; G01N 33/68; G01N 1/40; G01N 33/02; G01N 33/00

(52) U.S. Cl. CPC G01N 1/286; G01N 33/54366; G01N 33/5304; G01N 2001/2866; G01N 1/4044; G01N 33/025; G01N 33/0098; G01N 33/6854

(57) ABSTRACT

The present invention relates to assays and methods for detecting the presence, absence and/or amount of an allergen (e.g., a food allergen) in a sample. The current assays and methods improve sampling processes, increase detection accuracy and sensitivity, and reduce the detection time.

### CROSS REFERENCES TO RELATED APPLICATIONS

This application claims the benefit of priority of U.S. Provisional Application Ser. No. 62/305,134 filed on Mar. 8, 2016; U.S. Provisional Application Ser. No. 62/187,871 filed on Jul. 2, 2015; and U.S. Provisional Application Ser. No. 62/182,718 filed on Jun. 22, 2015; the contents of each of which are incorporated herein by reference in their entirety.

### FIELD OF THE INVENTION

The present invention relates to methods and assays optimized for detecting the presence, absence and/or amount of an allergen (e.g., a food allergen) in a sample. In particular, the detection methods may be employed in portable devices designed for allergen detection.

### BACKGROUND OF THE INVENTION

Allergy is a serious medical condition affecting millions of people worldwide, with about 15 million people in the United States, including many children. Allergic reactions range from mild to severe or life-threatening. Severe symptoms may include difficulty in breathing, low blood pressure, chest pain, loss of consciousness, and anaphylaxis. Food allergies are a major health issue in all industrialized countries. People having food allergies currently manage their allergies by avoiding any food that might contain that specific allergen. These restrictions have a major impact on the patients' quality of life and there remains no method for assessing the true allergen content of food. In the United States, food allergy symptoms send someone to the emergency room every three minutes.

Allergen detection is important for many reasons. A fast and accurate detection method and a portable device that can be easily operated by a person with food allergies will benefit the person to determine accurately and immediately the allergen content before consuming a food product. In food industry, allergen detection is critical to ensure accuracy of food labeling and to clean contaminants effectively during food production.

Portable devices for allergen detection are available or under development in the field. Some examples include strip test kits, immunological biosensors, microfluidic chips by Jung et al., (US Patent Publication NO.: 2008/0182339 and U.S. Pat. No. 8,617,903), a portable detection device by Scott (US Patent Publication NO.: 2010/0210033), a food test device by Royds (U.S. Pat. No. 7,527,765), a self-testing allergen test kit disclosed in PCT publication No. 2012/078455; and portable detection systems by Sundvor et al., (US Patent Publication NOs.: 2014/0295406 and 2015/0011020; PCT publication NO: 2014/160861; and PCT application NO. PCT/US14/57776).

Currently available assays/methods employed in these detection devices sometimes are very complex, expensive, time consuming and unreliable. There are several drawbacks in most of current detection methods. These methods lack an optimized sampling mechanism that can measure an adequate amount of test sample, and sample processing procedures that can ensure maximal extraction of allergen proteins from test samples. The accuracy and sensitivity of allergen detection varies in most methods, particularly in many antibody-based methods. The time to complete a testing assay can range from more than 10 minutes to several hours. For example, an ELISA assay can take about 30 minutes in a fast ELISA assay and about 3 hours in a standard ELISA assay. Additionally, some detection methods only generate qualitative analysis such as positive dots, lines and bands as indication of the presence of an allergen in a sample. Such qualitative analysis sometime is not valuable and a quantitative analysis to detect the amount of an allergen in a sample is important in some circumstances. In one particular example, in the strip detection substrate of Sundvor's portable detection device, (U.S. Patent Publication NOs.: 2014/0295406 and 2015/0011020 and PCT Patent Publication NO.: 2014/160861, the content of each of which is herein incorporated by reference in their entirety), the sample dispersion is mixed with complementary antibodies bound to the detection substrates, and the interaction between allergen and antibodies is indicated as positive dots, bands or lines. However, parameters that might affect the sensitivity and repeatability are not taken into account and optimized to obtain the best detection result.

A fast and accurate detection of the presence, absence and/or amount of an allergen protein in a food sample would be of great benefit. For instance, in restaurant, the restaurant owner or a consumer needs to know if a particular allergen is present (or the amount) in a cooked dish in minutes. A testing that will take hours to complete would be disadvantageous. The present disclosure provides methods and assays that improve the sampling mechanisms and accelerate allergen extraction process from a sample with high efficacy. Furthermore, the present invention develops steps and improvements to increase accuracy and sensitivity of a detection assay. Such methods and assays will improve the performance of a detection device, particularly a portable device for individual use.

### SUMMARY OF THE INVENTION

The present invention provides improved methods for allergen detection. In some embodiments, the present invention provides assays and methods for detecting the absence, presence and/or amount of one or more allergens in a food sample. In some cases, these methods comprise the steps of: (a) obtaining a test sample; (b) receiving the test sample in a first chamber of a test container in a detection device through a reception port of the first chamber configured to receive the test sample; (c) processing the test sample in the first chamber by homogenization in an extraction buffer; (d) delivering the processed sample to a second chamber of the test container through a second port of the first chamber, wherein said second port is configured to deliver the processed sample in the first chamber to the second chamber, and wherein the second chamber is configured to receive the processed sample from the second port of the first chamber; (e) contacting the processed sample with one or more detection agent, wherein said one or more detection agent specifically binds to said allergen; and (f) observing a detection signal.

In some cases, methods of the invention further comprise pre-processing test samples. Such pre-processing may include cutting into small pieces, crushing into powder, smashing into small particles, heating, or a combination thereof. In further cases, extraction buffer may be optimized to achieve a maximal extraction of proteins from test samples.

In some embodiments, step (e) further comprises providing one or more detection agent to the analytic chamber within the test container. In some cases, detection agents may be confined to a surface in the analytic chamber within the test container. Surfaces may be test strips, membranes on a test strip, plastic surfaces, and glass surfaces.

In some embodiments, detection agents may comprise one or more antibody which specifically binds to a target allergen. Such antibodies may be selected from monoclonal antibodies, polyclonal antibodies, antibody fragments, and antibody variants. In some cases, antibodies may comprise a detectable label. Detectable labels may be selected from fluorescent labels, luminescent labels, enzymatic labels, and radioactive labels. In some cases, binding of antibodies to allergens may be detected with secondary antibodies. Such secondary antibodies may comprise detectable labels.

In some embodiments, processed samples from the first chamber may be delivered to the analytic chamber by a means selected from air pressure, vacuum pressure, filtration, and diaphragm pump. In some cases, first chambers may comprise a homogenizer configured for homogenizing test samples and may be controlled by a motor within the detection devices. Homogenizers may be provided with a heating and/or cooling mechanism.

Extraction buffer may be stored in the first chamber and contacted with test sample during homogenization or extraction buffer may be provided to the first chamber during homogenization.

Detected allergens may be selected from the group consisting of a food allergen, an allergen from the environment, and a medical allergen. In some cases, food allergens may be allergenic proteins associated with food. In some cases, these may be allergens associated with peanuts, such as Ara h 1, Ara h 2, Ara h 3, Ara h 4, Ara h 5, Ara h 6, Ara h 7, Ara h 8, Ara h 9, Ara h 10, Ara h 11, Ara h 12, Ara h 13.

In some embodiments, methods of the invention may be carried out in less than 5 minutes.

In some embodiments, one or more standard samples may also be analyzed and results may be compared to those obtained from analysis of processed samples. Such comparisons may be used to obtain the level of allergen in such processed samples. In some cases, test samples may comprise less than 0.1% concentration of allergen.

### BRIEF DESCRIPTION OF THE DRAWINGS

FIG. 1 represents a PRIOR ART figure from US patent publication NO. US2015/0011020, FIG. 1.

FIG. 2 represents a PRIOR ART figure from US patent publication NO. US2015/0011020, FIG. 2A.

FIG. 3 represents a PRIOR ART figure from US patent publication NO. US2015/0011020, FIG. 2B.

FIG. 4A represents a PRIOR ART figure from US patent publication NO. US2015/0011020, FIG. 4A; and FIG. 4B represents a PRIOR ART figure from US patent publication NO. US2015/0011020, FIG. 4B.

FIG. 5 represents a PRIOR ART figure from US patent publication NO. US2015/0011020, FIG. 10.

### DETAILED DESCRIPTION OF THE INVENTION

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

In order to assist in the control of allergen levels in foods to acceptable levels and the decision whether it is safe to consume a food product or not, a test method/assay, which is designed to provide accurate, timely, and cost-effective analytical information, will be beneficial. The present invention provides devices and methods for detecting allergens using one or more detection agents.

In some embodiments, the present invention combines detection agents described herein with the portable detection devices of US Patent Publication NOs.: 2014/0295406 and 2015/0011020, the contents of each of which are herein incorporated by reference in their entirety.

Currently available tests are often slow and/or inaccurate. Strip tests are antibody-based tests that are easy to use and fast but usually take more than 10 minutes to complete. Results are only qualitative or semi-quantitative. Enzyme-linked immunosorbant assays are also simple antibody-based methods known in the art. The analysis time of an ELISA assay ranges from 30 minutes for fast ELISAs to three hours for standard ELISAs. Even for recently developed biosensor detection assays, the assay time may be more than 10 minutes. There are factors that may affect the antibody-based methods such as food matrices (e.g., heated food or cooked food), allergen processing agents (e.g., extraction buffer) and procedures, antibody specificity (e.g. cross reaction between different allergens) and sensitivity, and detection method. The present invention optimizes sample processing procedures to reduce the reaction time, and to increase reaction sensitivity and reliability. A detailed description of detection agents; allergens that can be detected by current methods/assays; improved processing steps; and detection methods are presented below.

In some embodiments, methods and assays of the present invention are accurate, timely and sensitive in detecting an allergen in a sample. In particle, devices and methods of the invention yield fast detection results, making them useful for an individual looking for an immediate indication of the allergen content of a sample (e.g., a food or beverage).

### Detection Agents

In order to detect allergens in samples, an agent or molecule that can specifically bind to such allergens is important. As used herein, the term “detection agent” is used to refer to such agents or molecules. Binding between a detection agent and a target allergen can then be used to generate a signal (e.g., fluorescent, colorimetric, enzymatic, luminescent, radioactive, etc.) to indicate binding, referred to herein as a “detection signal.” The source of the detection signal can include a conjugate (e.g., fluorescent conjugate, colorimetric conjugate, enzymatic conjugate, luminescent conjugate, radioactive conjugate, etc.) to the detection agent itself or to a second agent (e.g., a secondary antibody) that recognizes the bound detection agent.

Detection signals may be observed through any means known in the art. As such, observing a detection signal may include, but is not limited to, spectrophotometric detection, fluorescence detection (e.g., using a fluorometric device), luminometric detection (e.g., using a luminometer), radiometric detection (e.g., through the use of a radioactivity-sensitive film or emulsion; scintillation counter; giger counter; and the like), and visualization (e.g., a perceivable change to a test strip, such as a color change).

In some embodiments, the level or intensity of a detection signal may be used to quantify the level of one or more allergens in a test sample. In such cases, detection signals may be compared to detection signals obtained using one or more standard samples. As used herein, the term “standard sample” refers to a sample with known levels of one or more allergens or allergen surrogate compounds (alternate compounds designed to bind to the same detection agent as a particular allergen, e.g., an allergen fragment fused or otherwise attached to another compound). Analysis of standard samples may be used to generate a standard curve that can be used to elucidate test sample allergen levels by comparison. Analysis of such standard samples may further be used to determine the limits for detecting allergens in samples with low or high allergen concentrations.

Detection agents may include, but are not limited to antibodies, proteins, nucleic acids, small molecules, macromolecules, and dendrimers. As used herein, the term “target allergen” refers to a specific allergen, fragment thereof, or allergen compound recognized by a specific detection agent.

Detection agents may be used to detect any allergens, such allergens including, but not limited to food allergens; allergens from the environment (e.g., from animals, plants, microbes, air, and water), and medical allergens. Detection agents may further be used to detect any allergens taught herein.

### Antibodies

In some embodiments, the detection agents of the present invention may comprise antibodies. As used herein, the term “antibody” is used in the broadest sense and specifically includes (but is not limited to) whole antibodies, monoclonal antibodies, polyclonal antibodies, multispecific antibodies (e.g. bispecific antibodies formed from at least two intact antibodies), antibody fragments, diabodies, antibody variants, and antibody-derived binding domains that are part of or associated with other peptides. Antibodies are primarily amino-acid based molecules but may also comprise one or more modifications (including, but not limited to the addition of sugar moieties, fluorescent moieties, chemical tags, etc.).

As used herein the term, “antibody fragment” refers to any portion of an intact antibody. In some embodiments, antibody fragments comprise antigen binding regions from intact antibodies. Examples of antibody fragments may include, but are not limited to Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Compounds and/or compositions of the present invention may comprise one or more of these fragments. For the purposes herein, an “antibody” may comprise a heavy and light variable domain as well as an Fc region.

As used herein, the term “native antibody” refers to a usually heterotetrameric glycoprotein of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains that differ extensively in sequence among antibodies and are used in the binding and specificity of each particular antibody for its particular antigen.

As used herein, the term “Fv” refers to antibody fragments comprising complete antigen-recognition and antigen-binding sites. These regions consist of a dimer of one heavy chain and one light chain variable domain in tight, non-covalent association.

As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “Single-chain Fv” or “scFv” refers to a fusion protein of V_{H} and V_{L} antibody domains, wherein these domains are linked together into a single polypeptide chain. In some embodiments, the Fv polypeptide linker enables the scFv to form the desired structure for antigen binding.

As used herein, the term “bispecific antibody” refers to an antibody capable of binding two different antigens. Such antibodies typically comprise regions from at least two different antibodies. Bispecific antibodies may include any of those described in Riethmuller, G. 2012, Cancer Immunity, 12:12-18; Marvin, J. S. et al., 2005, Acta Pharmacologica Sinica. 26(6):649-58 and Schaefer, W. et al., 2011, PNAS, 108(27):11187-92; the contents of each of which are herein incorporated by reference in their entirety.

As used herein, the term “diabody” refers to a small antibody fragment with two antigen-binding sites. Diabodies comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (Hollinger, P. et al., “Diabodies”: Small bivalent and bispecific antibody fragments, PNAS, 1993, 90:6444-6448) the contents of each of which are incorporated herein by reference in their entirety.

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity.

As used herein, the term “hypervariable region” refers to regions within the antigen binding domain of an antibody comprising amino acid residues responsible for antigen binding. The amino acids present within the hypervariable regions determine the structure of the complementarity determining region (CDR). As used herein, the term “CDR” refers to regions of antibodies comprising a structure that is complimentary to its target antigen or epitope.

In some embodiments, compounds and/or compositions of the present invention may be antibody mimetics. As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. No. 6,673,901; U.S. Pat. No. 6,348,584). In some embodiments, antibody mimetics may include any of those known in the art including, but are not limited to affibody molecules, affilins, affitins, anticalins, avimers, Centyrins, DARPINS™, Fynomers and Kunitz and domain peptides. In other embodiments, antibody mimetics may include one or more non-peptide region.

As used herein, the term “antibody variant” refers to a biomolecule resembling an antibody in structure and/or function comprising some differences in their amino acid sequence, composition or structure as compared to a native antibody.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012, the contents of which are herein incorporated by reference in their entirety.

### Antibody Development

Antibodies of the invention may be purchased from commercial sources or may be produced through any methods known in the art. Such methods may include, but are not limited to recombinant synthesis; mutation or optimization of a known antibody; selection from a an antibody library or antibody fragment library; and immunization.

Methods of antibody development typically rely on the use of a target molecule for selection, immunization, and/or confirmation of antibody affinity and/or specificity. Target molecules used according to the present invention include target allergens.

Target allergens may be amino acid-based molecules, non-amino acid based molecules, or compounds made up of both amino acid-based molecules and non-amino acid-based molecules.

The terms “amino acid” and “amino acids” refer to all naturally occurring L-alpha-amino acids as well as non-naturally occurring amino acids. Amino acids are identified by either the one-letter or three-letter designations as follows: aspartic acid (Asp: D), isoleucine (Ile: I), threonine (Thr: T), leucine (Leu: L), serine (Ser: S), tyrosine (Tyr: Y), glutamic acid (Glu: E), phenylalanine (Phe: F), proline (Pro: P), histidine (His: H), glycine (Gly: G), lysine (Lys: K), alanine (Ala: A), arginine (Arg: R), cysteine (Cys: C), tryptophan (Trp: W), valine (Val: V), glutamine (Gln: Q) methionine (Met: M), and asparagine (Asn: N), where the amino acid is listed first followed parenthetically by the three and one letter codes, respectively.

Amino acid-based target allergens may be proteins or peptides. As used herein, the term “peptide” refers to an amino-acid based molecule having from 2 to 50 or more amino acids. Special designators apply to the smaller peptides with “dipeptide” referring to a two amino acid molecule and “tripeptide” referring to a three amino acid molecule. Amino acid based molecules having more than 50 contiguous amino acids are considered polypeptides or proteins.

### Immunization Methods

In some embodiments, antibodies can be prepared through immunization of a host with one or more target allergens, which act as immunogens to elicit an immunological response. In some cases, only portions or regions of a given allergen may be used. In the case of amino acid-based allergens, one or more allergen-derived peptides (referred to herein as “allergen peptides”) may be used. Allergen peptides suitable for generating antibodies preferably contain a sequence of at least 4, at least 5, at least 6, at least 7, at least 8, at least 9, at least 10, at least 11, at least 12, at least 13, at least 14, at least 15, from about 5 to about 50 amino acids, from about 10 to about 30 amino acids, from about 10 to about 20 amino acids, from about 40 to about 200 amino acids, or at least 200 amino acids in length.

In certain embodiments of the present invention, where larger polypeptides or proteins are used for generating antibodies, these preferably are at least 50, at least 55, at least 60, at least 70, at least 80, at least 90, or more amino acids in length.

### Immunogenic Hosts

Antibody generation by immunization typically involves the use of non-human animal hosts as subjects for immunization, referred to herein as “immunogenic hosts.” In some embodiments, immunogenic hosts are selected from any vertebrates. In further embodiments, immunogenic hosts are selected from all mammals. In further embodiments, immunogenic hosts are mice, including transgenic or knockout mice. Other immunogenic hosts may include, but are not limited to rats, rabbits, cats, dogs, goats, sheep, hamsters, guinea pigs, cows, horses, pigs, llamas, camels, and chickens.

### Adjuvants

Immunization of immunogenic hosts with target allergens described herein may comprise the use of one or more adjuvants. Adjuvants may be used to elicit a higher immune response in such immunogenic hosts. As such, adjuvants used according to the present invention may be selected based on their ability to affect antibody titers. Adjuvants may include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and other useful human adjuvants such as BCG (Bacillus Calmette-Guerin) and Corynebacterium parvum. Such adjuvants are also well known in the art.

In some embodiments, water-in-oil emulsions may be useful as adjuvants. Water-in-oil emulsions may act by forming mobile antigen depots, facilitating slow antigen release and enhancing antigen presentation to immune components. Freund's adjuvant may be used as complete Freund's adjuvant (CFA), which comprises mycobacterial particles that have been dried and inactivated, or as incomplete Freund's adjuvant (IFA), lacking such particles. Other water-in-oil-based adjuvants may include EMULSIGEN® (MVP Technologies, Omaha, Nebr.). EMULSIGEN® comprises micron sized oil droplets that are free from animal-based components. It may be used alone or in combination with other adjuvants, including, but not limited to aluminum hydroxide and CARBIGEN™ (MVP Technologies, Omaha, Nebr.).

In some embodiments, TITERMAX® adjuvant may be used. TITERMAX® is another water-in-oil emulsion comprising squalene as well as sorbitan monooleate 80 (as an emulsifier) and other components. In some cases, TITERMAX® may provide higher immune responses, but with decreased toxicity toward immunogenic hosts.

Immunostimmulatory oligonucleotides may also be used as adjuvants. Such adjuvants may include CpG oligodeoxynucleotide (ODN). CpG ODNs are recognized by Toll-like receptor 9 (TLR9) leading to strong immunostimulatory effects. Type C CpG ODNs induce strong IFN-α production from plasmacytoid dendritic cell (pDC) and B cell stimulation as well as IFN-γ production from T-helper (Tx) cells. CpG ODN adjuvant have been shown to significantly enhance pneumococcal polysaccharide (19F and type 6B)-specific IgG2a and IgG3 in mice. CpG ODN also enhanced antibody responses to the protein carrier CRM197, particularly CRM197-specific IgG2a and IgG3 (Chu et al., Infection Immunity 2000, vol 68(3):1450-6). Additionally, immunization of aged mice with pneumococcal capsular polysaccharide serotype 14 (PPS14) combined with a CpG-ODN restored IgG anti-PPS14 responses to young adult levels (Sen et al., Infection Immunity, 2006, 74(3):2177-86). CpG ODNs used according to the present invention may include class A, B or C ODNs. In some embodiments, ODNs may include any of those available commercially, such as ODN-1585, ODN-1668, ODN-1826, ODN-2006, ODN-2007, ODN-2216, ODN-2336, ODN-2395 and/or ODN-M362, each of which may be purchased, for example, from InvivoGen, (San Diego, Calif.). In some cases, ODN-2395 may be used. ODN-2395 is a class C CpG ODN that specifically stimulated human as well as mouse TLR9. These ODNs comprise phosphorothioate backbones and CpG palindromic motifs.

In some embodiments, immune stimulating complexes (ISCOMs) may be used as adjuvants. ISCOMs are spherical open cage-like structures (typically 40 nm in diameter) that are spontaneously formed when mixing together cholesterol, phospholipids and Quillaia saponins under a specific stoichiometry. ISCOM technology is proven for a huge variety of antigens from large glycoproteins such as gp340 from Epstein-Barr virus (a 340 kDa antigen consisting of 80% carbohydrates) down to carrier-conjugated synthetic peptides and small haptens such as biotin. Some ISCOMs are capable of generating a balanced immune response with both T_{H1} and T_{H2} characteristics. Immune response to ISCOMs is initiated in draining lymph nodes, but is efficiently relocated to the spleen, which makes it particularly suitable for generating monoclonal antibodies as well. In some embodiments, the ISCOM adjuvant AbISCO-100 (Isconova, Uppsala, Sweden) may be used. AbISCO-100 is a saponin-based adjuvant specifically developed for use in immunogenic hosts, such as mice, that may be sensitive to other saponins.

According to embodiments of the present invention, adjuvant components of immunization solutions may be varied in order to achieve desired results. Such results may include modulating the overall level of immune response and/or level of toxicity in immunogenic hosts.

### Monoclonal Antibody Production

Monoclonal antibodies of the present invention can be prepared using well-established methods known by those skilled in the art. In one embodiment, the monoclonal antibodies are prepared using hybridoma technology (Kohler, G. et al., Continuous cultures of fused cells secreting antibody of predefined specificity. Nature. 1975 Aug. 7: 256 (5517): 495-7). In a hybridoma method, a mouse, hamster, or other appropriate immunogenic host animal, is typically immunized with an immunizing agent (e.g., a target allergen) to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell (Goding, J. W., Monoclonal Antibodies: Principles and Practice. Academic Press. 1986; 59-1031). Immortalized cell lines are usually transformed mammalian cells, particularly myeloma cells of rodent, rabbit, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, D. et al., A human hybrid myeloma for production of human monoclonal antibodies. J Immunol. 1984 December; 133(6): 3001-5; Brodeur, B. et al., Monoclonal Antibody Production Techniques and Applications. Marcel Dekker, Inc., New York. 1987; 33:51-63).

The culture medium in which the hybridoma cells are cultured can then be assayed for the presence of monoclonal antibodies. Preferably, the binding specificity (i.e., specific immunoreactivity) of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known by those skilled in the art. The binding specificity of the monoclonal antibody can, for example, be determined by Scatchard analysis (Munson, P. J. et al., Ligand: a versatile computerized approach for characterization of ligand-binding systems. Anal Biochem. 1980 Sep. 1; 107(1):220-39).

After the desired hybridoma cells are identified, the clones may be subcloned by limiting dilution procedures and grown by standard methods. Suitable culture media for this purpose include, for example, Dulbecco's Modified Eagle's Medium or RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In another embodiment, the monoclonal antibodies of the present invention can also be made by recombinant DNA methods, such as those described in U.S. Pat. No. 4,816,567, which is hereby incorporated by reference in its entirety. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567) or by covalently joining the immunoglobulin coding sequence with all or part of the coding sequence for a non-immunoglobulin polypeptide. Such a non-immunoglobulin polypeptide can be substituted for the constant domains of an antibody of the invention, or can be substituted for the variable domains of an antibody of the invention to create a chimeric bivalent antibody.

### Polyclonal Antibody Production

Antibodies of the present invention can also be produced by various procedures known by those skilled in the art for the production of polyclonal antibodies. Polyclonal antibody production typically involves immunization of immunogenic host animals, such as rabbits, rats, mice, sheep, or goats, with either free or carrier-coupled immunogens (e.g., target allergens), for example, by intraperitoneal and/or intradermal injection. Injection material is typically an emulsion containing about 100 μg of immunogen or carrier protein. Several booster injections may be needed, for instance, at intervals of about two weeks, to provide a useful titer of antibody which can be detected, for example, by ELISA assay using free peptide adsorbed to a solid surface. The titer of antibodies in serum from an immunized animal can be increased by selection of antibodies, e.g., by adsorption of the peptide onto a solid support and elution of the selected antibodies according to methods well known in the art.

### Antibody Selection

A desired antibody may be selected from a larger pool of two or more candidate antibodies based on affinity and/or specificity for target allergens and/or epitopes thereof. In some embodiments, antibody selection may be carried out using an antibody binding assay. Such assays may include, but are not limited to surface plasmon resonance (SPR)-based assays, ELISAs, and flow cytometry-based assays. Assays may utilize a target allergen to bind a desired antibody and then use one or more detection methods to detect binding.

In some embodiments, antibodies of the invention may be selected and produced using high throughput methods of discovery. In one embodiment, antibodies of the invention are produced through the use of display libraries. The term “display” as used herein, refers to the expression or “display” of proteins or peptides on the surface of a given display host. The term “library” as used herein, refers to a collection of unique cDNA sequences. A library may contain from as little as two unique cDNAs to hundreds of billions of unique cDNAs. In some embodiments, detection agents comprising synthetic antibodies are produced using antibody display libraries or antibody fragment display libraries. The term “antibody fragment display library” as used herein, refers to a display library wherein each member encodes an antibody fragment containing at least one variable region of an antibody. Such antibody fragments are preferably Fab fragments, but other antibody fragments such as single-chain variable fragments (scFvs) are contemplated as well. In a Fab antibody fragment library, each Fab encoded may be identical except for the amino acid sequence contained within the variable loops of the complementarity determining regions (CDRs) of the Fab fragment. In an alternative or additional embodiment, amino acid sequences within the individual VH and/or VL regions may differ as well.

Display libraries may be expressed in a number of possible hosts (referred to herein as “display hosts”) including, but not limited to yeast, bacteriophage (also referred to herein as “phages” or “phage particles,” bacteria and retroviruses. Additional display technologies that may be used include ribosome-display, microbead-display and protein-DNA linkage techniques. When expressed, the Fabs decorate the surface of the host (e.g., phage or yeast) where they can interact with a given target allergen. Any target allergens may be used to select display hosts expressing antibody fragments with the highest affinity for that target. The DNA sequence encoding the variable domains of the bound antibody fragment can then be determined through sequencing using the bound particle or cell. In one embodiment, positive selection is used in the development of antibodies. As used herein, the term “positive selection” refers to processes by which antibodies and/or fragments thereof are selected from display libraries based on affinity for target allergens containing desirable target sites. In some embodiments, negative selection is utilized in the development of antibodies. As used herein, the term “negative selection” refers to processes by which non-target agents are used to exclude antibodies and/or fragments thereof from a given display library during antibody development. In some embodiments, both positive and negative selection processes are utilized during multiple rounds of selection in the development of antibodies using display libraries.

In yeast display, cDNA encoding different antibody fragments are introduced into yeast cells where they are expressed and the antibody fragments are “displayed” on the cell surface as described by Chao et al. (Chao, G. et al., Isolating and engineering human antibodies using yeast surface display. Nat Protoc. 2006; 1(2):755-68). In yeast surface display, expressed antibody fragments contain an additional domain comprising the yeast agglutinin protein, Aga2p. This domain allows the antibody fragment fusion protein to attach to the outer surface of the yeast cell through the formation of disulfide bonds with surface-expressed Aga1p. The result is a yeast cell, coated in a particular antibody fragment. Display libraries of cDNA encoding these antibody fragments are utilized initially in which the antibody fragments each have a unique sequence. These fusion proteins are expressed on the cell surface of millions of yeast cells where they can interact with a desired targets, incubated with the cells. Target peptides may be covalently or otherwise modified with a chemical or magnetic group to allow for efficient cell sorting after successful binding with a suitable antibody fragment takes place. Recovery may be by way of magnetic-activated cell sorting (MACS), fluorescence-activated cell sorting (FACS) or other cell sorting methods known in the art. Once a subpopulation of yeast cells is selected, the corresponding plasmids may be analyzed to determine the sequence of displayed antibody fragments.

Bacteriophage display methods typically utilize filamentous phage including fd, F1 and M13 virions. Such strains are non-lytic, allowing for continued propagation of the host and increased viral titres. Examples of phage display methods that can be used to make the antibodies of the present invention include those disclosed in Miersch et al. (Miersch, S. et al., Synthetic antibodies: Concepts, potential and practical considerations. Methods. 2012 August; 57(4): 486-98), Bradbury et al. (Bradbury, A. R. et al., Beyond natural antibodies: the power of in vitro display technologies. Nat Biotechnol. 2011 March; 29(3):245-54), Brinkman et al. (Brinkmann, U. et al., Phage display of disulfide-stabilized Fv fragments. J Immunol Methods. 1995 May 11; 182(1):41-50); Ames et al. (Ames, R. S. et al., Conversion of murine Fabs isolated from a combinatorial phage display library to full length immunoglobulins. J Immunol Methods. 1995 Aug. 18; 184(2):177-86); Kettleborough et al. (Kettleborough, C. A. et al., Isolation of tumor cell-specific single-chain Fv from immunized mice using phage-antibody libraries and the re-construction of whole antibodies from these antibody fragments. Eur J Immunol. 1994 April; 24(4):952-8); Persic et al. (Persic, L. et al., An integrated vector system for the eukaryotic expression of antibodies or their fragments after selection from phage display libraries. Gene. 1997 Mar. 10; 187(1):9-18); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5, 969,108, each of which is incorporated herein by reference in its entirety.

Antibody fragment expression on bacteriophages may be carried out by inserting the cDNA encoding the fragment into the gene expressing a viral coat protein. The viral coat of filamentous bacteriophages is made up of five coat proteins, encoded by a single-stranded genome. Coat protein pIII is the preferred protein for antibody fragment expression, typically at the N-terminus. If antibody fragment expression compromises the function of pIII, viral function may be restored through coexpression of a wild type pIII, although such expression will reduce the number of antibody fragments expressed on the viral coat, but may enhance access to the antibody fragment by the target. Expression of viral as well as antibody fragment proteins may alternatively be encoded on multiple plasmids. This method may be used to reduce the overall size of infective plasmids and enhance the transformation efficiency.

Phage display libraries may comprise millions to billions of phage particles, each expressing unique antibody fragments on their viral coats. Such libraries may provide richly diverse resources that may be used to select potentially hundreds of antibody fragments with diverse levels of affinity for one or more targets (McCafferty, et al., 1990. Nature. 348:552-4; Edwards, B. M. et al., 2003. JMB. 334: 103-18; Schofield, D. et al., 2007. Genome Biol. 8, R254 and Pershad, K. et al., 2010. Protein Engineering Design and Selection. 23:279-88; the contents of each of which are herein incorporated by reference in their entirety). Often, the antibody fragments present in such libraries comprise scFv antibody fragments, comprising a fusion protein of VH and VL antibody domains joined by a flexible linker (e.g. a Ser/Gly-rich linker). These fragments typically comprise the VH domain first, but VL-linker-VH fragments are also contemplated herein. In some cases, scFvs may contain the same sequence with the exception of unique sequences encoding variable loops of the complementarity determining regions (CDRs). In some cases, scFvs are expressed as fusion proteins, linked to viral coat proteins (e.g. the N-terminus of the viral pIII coat protein). VL chains may be expressed separately for assembly with VH chains in the periplasm prior to complex incorporation into viral coats.

In some cases, phage enrichment comprises solution-phase phage enrichment where target allergens are present in a solution that is combined with phage solutions. According to such methods, target allergens may comprise detectable labels (e.g. biotin labels) to facilitate retrieval from solution and recovery of bound phage. In other embodiments, solution-phase phage enrichment may comprise the use of targets bound to beads (e.g. streptavidin beads). In some cases, such beads may be magnetic beads to facilitate precipitation.

In some embodiments, phage enrichment may comprise solid-phase enrichment where target allergens are immobilized on solid surfaces. According to such methods, phage solutions may be used to contact the solid surface for enrichment with the immobilized targets. Solid surfaces may include any surfaces capable of retaining targets and may include, but are not limited to dishes, plates, flasks, membranes, and tubes. In some cases, immunotubes may be used wherein the inner surface of such tubes are coated with target allergens (e.g. by passing biotinylated targets through streptavidin or neutravidin-coated tubes). Phage enrichment with immunotubes may be carried out by passage of phage solution through the tubes to enrich bound targets.

After selection, bound phage may be used to infect E. coli cultures that are co-infected with helper phage, to produce an amplified output library for the next round of enrichment. This process may be repeated producing narrower and narrower clone sets. In some embodiments, rounds of enrichment are limited to improve the diversity of selected phage.

Precipitated library members may be sequenced from the bound phage to obtain cDNA encoding desired scFvs. Such sequences may be directly incorporated into antibody sequences for recombinant antibody production, or mutated and utilized for further optimization through in vitro affinity maturation.

IgG antibodies comprising one or more variable domains from selected scFvs may be synthesized for further testing and/or product development. Such antibodies may be produced by insertion of one or more segments of scFv cDNA into expression vectors suited for IgG production.

### Antibody Engineering

After isolation or selection of target allergen-specific antibodies, antibody sequences may be used for recombinant production and/or optimization of such antibodies. In the case of antibody fragment isolation from a display library, coding regions from the isolated fragment may be used to generate whole antibodies, including human antibodies, or any other desired target binding fragment, and expressed in any desired host, including mammalian cells, insect cells, plant cells, yeast, and bacteria, e.g., as described in detail below.

Examples of techniques that can be used to produce antibodies and antibody fragments, such as Fabs and scFvs, include those described in U.S. Pat. Nos. 4,946,778 and 5,258,498; Miersch et al. (Miersch, S. et al., Synthetic antibodies: Concepts, potential and practical considerations. Methods. 2012 August; 57(4):486-98), Chao et al. (Chao, G. et al., Isolating and engineering human antibodies using yeast surface display. Nat Protoc. 2006; 1(2):755-68), Huston et al. (Huston, J. S. et al., Protein engineering of single-chain Fv analogs and fusion proteins. Methods Enzymol. 1991; 203:46-88); Shu et al. (Shu, L. et al., Secretion of a single-gene-encoded immunoglobulin from myeloma cells, Proc. Natl. Acad. Sci. U S. A., 1993 Sep. 1, 90(17):7995-7999); and Skerra et al. (Skerra, A. et al., Assembly of a functional immunoglobulin Fv fragment in Escherichia coli, Science, 1988 May 20, 240(4855):1038-1041); the contents of each of which are incorporated herein by reference in their entirety.

IgG antibodies (e.g. IgG1, IgG2, IgG3 or IgG4) may be synthesized for further testing and/or product development from variable domain fragments produced or selected according to the methods described herein. Such antibodies may be produced by insertion of one or more segments of cDNA encoding desired amino acid sequences into expression vectors suited for IgG production. Expression vectors may comprise mammalian expression vectors suitable for IgG expression in mammalian cells. Mammalian expression of IgGs may be carried out to ensure that antibodies produced comprise modifications (e.g. glycosylation) characteristic of mammalian proteins and/or to ensure that antibody preparations lack endotoxin and/or other contaminants that may be present in protein preparations from bacterial expression systems.

### Affinity Maturation

The DNA sequence encoding an antibody of the invention can be mutated for additional rounds of selection in a process known as affinity maturation. The term “affinity maturation,” as used herein, refers to a method whereby antibodies are produced with increasing affinity for a given target through successive rounds of mutation and selection of antibody- or antibody fragment-encoding cDNA sequences. In some cases, this process is carried out in vitro. To accomplish this, amplification of variable domain sequences (in some cases limited to CDR coding sequences) may be carried out using error-prone PCR to produce millions of copies containing mutations including, but not limited to point mutations, regional mutations, insertional mutations and deletional mutations. As used herein, the term “point mutation” refers to a nucleic acid mutation in which one nucleotide within a nucleotide sequence is changed to a different nucleotide. As used herein, the term “regional mutation” refers to a nucleic acid mutation in which two or more consecutive nucleotides are changed to different nucleotides. As used herein, the term “insertional mutation” refers to a nucleic acid mutation in which one or more nucleotides are inserted into a nucleotide sequence. As used herein, the term “deletional mutation” refers to a nucleic acid mutation in which one or more nucleotides are removed from a nucleotide sequence. Insertional or deletional mutations may include the complete replacement of an entire codon or the change of one codon to another by altering one or two nucleotides of the starting codon.

Mutagenesis may be carried out on CDR-encoding cDNA sequences to create millions of mutants with singular mutations in heavy and light chain CDR regions. In another approach, random mutations are introduced only at CDR residues most likely to improve affinity. These newly generated mutagenic libraries can be used to repeat the process to screen for clones that encode antibody fragments with even higher affinity for the target peptide. Continued rounds of mutation and selection promote the synthesis of clones with greater and greater affinity (Chao, G. et al., Isolating and engineering human antibodies using yeast surface display. Nat. Protoc. 2006; 1(2):755-68).

Affinity matured clones may be selected based on affinity as determined by binding assay (e.g., FACS, ELISA, surface plasmon resonance, etc.). Select clones may then be converted to IgG and tested further for affinity and functional activity. In some cases, the goal of affinity optimization is to increase the affinity by at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100 fold, at least 500-fold or at least 1,000-fold as compared to the affinity of the original antibody. In cases where optimized affinity is less than desired, the process may be repeated.

### Chimeric and Humanized Antibodies

For some uses, including the in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal immunoglobulin and a human immunoglobulin constant region. Methods for producing chimeric antibodies are known in the art. (Morrison, S. L., Transfectomas provide novel chimeric antibodies. Science. 1985 Sep. 20; 229(4719):1202-7; Gillies, S. D. et al., High-level expression of chimeric antibodies using adapted cDNA variable region cassettes. J Immunol Methods. 1989 Dec. 20; 125(1-2):191-202; and U.S. Pat. Nos. 5,807,715; 4,816,567; and 4,816,397, which are incorporated herein by reference in their entirety).

Humanized antibodies are antibody molecules from non-human species that bind to the desired target and have one or more complementarity determining regions (CDRs) from the nonhuman species and framework regions from a human immunoglobulin molecule. Often, framework residues in the human framework regions are substituted with corresponding residues from the CDR and framework regions of the donor antibody to alter, preferably improve, target binding. These framework substitutions are identified by methods well known in the art, e.g., by modeling of the interactions of the CDR and framework residues to identify framework residues important for target binding, and by sequence comparison to identify unusual framework residues at particular positions. (U.S. Pat. Nos. 5,693,762 and 5,585,089; Riechmann, L. et al., Reshaping human antibodies for therapy. Nature. 1988 Mar. 24; 332(6162):323-7, which are incorporated herein by reference in their entireties).

Antibodies can be humanized using a variety of techniques known in the art, including, for example, CDR-grafting (EP 239,400; PCT publication WO 91/09967; U.S. Pat. Nos. 5,225,539; 5,530,101; and 5,585,089); veneering or resurfacing (EP 592,106; EP 519,596; Padlan, E. A., A possible procedure for reducing the immunogenicity of antibody variable domains while preserving their ligand-binding properties. Mol Immunol. 1991 April-May; 28 (4-5):489-98; Studnicka, G. M. et al., Human-engineered monoclonal antibodies retain full specific binding activity by preserving non-CDR complementarity-modulating residues. Protein Eng. 1994 June; 7(6):805-14; Roguska, M. A. et al., Humanization of murine monoclonal antibodies through variable domain resurfacing. Proc. Natl. Acad. Sci. U.S.A 1994 Feb. 1; 91(3):969-73); and chain shuffling (U.S. Pat. No. 5,565,332); each of which is incorporated herein by reference in their entirety.

Completely human antibodies are particularly desirable for therapeutic treatment of human patients, so as to avoid or alleviate immune reaction to foreign protein. Human antibodies can be made by a variety of methods known in the art, including the antibody display methods described above, using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin polynucleotides. For example, the human heavy and light chain immunoglobulin polynucleotide complexes can be introduced randomly, or by homologous recombination, into mouse embryonic stem cells. Alternatively, the human variable region, constant region, and diversity region may be introduced into mouse embryonic stem cells, in addition to the human heavy and light chain polynucleotides. The mouse heavy and light chain immunoglobulin polynucleotides can be rendered nonfunctional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected immunogen (e.g., target allergen).

Thus, using such a technique, it is possible to produce useful human IgG, IgA, IgM, IgD and IgE antibodies. For an overview of the technology for producing human antibodies, see Lonberg and Huszar (Lonberg, N. et al., Human antibodies from transgenic mice. Int. Rev. Immunol. 1995; 13(1):65-93). For a detailed discussion of the technology for producing human antibodies and human monoclonal antibodies and protocols for producing such antibodies, see, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598, each of which are incorporated by reference herein in their entirety. In addition, companies such as Abgenix, Inc. (Fremont, Calif.), Protein Design Labs, Inc. (Mountain View, Calif.) and Genpharm (San Jose, Calif.) can be engaged to provide human antibodies directed against a selected target using technology similar to the above described technologies.

Once an antibody molecule of the present invention has been produced by an animal, a cell line, chemically synthesized, or recombinantly expressed, it can be purified (i.e., isolated) by any method known in the art for the purification of an immunoglobulin or polypeptide molecule, for example, by chromatography (e.g., ion exchange, affinity, particularly by affinity for the specific target, Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

In accordance with the present invention, antibodies specifically binding to an allergen may be present in a solution or bound to a substrate. In some embodiments, the antibodies are bound to cellulose nanobeads and confined in one or more detection area of a substrate of a detection device.

### Antibody Characterization

Antibodies of the invention may be characterized by one or more of structure; isotype; binding (e.g., affinity and specificity); conjugate; glycosylation; or other distinguishing features.

Antibodies of the present invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), intracellularly made antibodies (i.e., intrabodies), and epitope-binding fragments of any of the above.

Antibodies of the present invention can be from any animal origin including birds and mammals. Preferably, such antibodies are of human, murine (e.g., mouse and rat), donkey, sheep, rabbit, goat, guinea pig, camel, horse, or chicken origin.

The antibodies of the present invention can be monospecific or multispecific (e.g., bispecific, trispecific, or of greater multispecificity). Multispecific antibodies can be specific for different epitopes of a peptide of the present invention, or can be specific for both a peptide of the present invention, and a heterologous epitope, such as a heterologous peptide or solid support material. (See, e.g., WO 93/17715; WO 92/08802; WO 91/00360; WO 92/05793; Tuft, A. et al., J Immunol. 1991 Jul. 1; 147(1):60-69; U.S. Pat. Nos. 4,474,893; 4,714,681; 4,925,648; 5,573,920; 5,601,819; and Kostelny, S. A. et al., J Immunol. 1992 Mar. 1; 148(5):1547-1553). For example, the antibodies may be produced against a peptide containing repeated units of a peptide sequence of the present invention, or they may be produced against a peptide containing two or more peptide sequences of the present invention, or the combination thereof. As a non-limiting example, a heterobivalent ligand (HBL) system that competitively inhibits allergen binding to mast cell bound IgE antibody, thereby inhibiting mast cell degranulation, has been designed (Handlogten, et al., Design of a Heterobivalent Ligand to Inhibit IgE Clustering on Mast Cells, Chemistry & Biology, 2011 Sep. 23, 18(9):1179-1188).

Antibody characteristics may be determined relative to a standard under normal physiologic conditions, in vitro or in vivo. Measurements may also be made relative to the presence or absence of the antibodies. Such methods of measuring include standard measurement in tissue or fluids such as serum or blood such as Western blot, enzyme-linked immunosorbent assay (ELISA), activity assays, reporter assays, luciferase assays, polymerase chain reaction (PCR) arrays, gene arrays, real time reverse transcriptase (RT) PCR and the like.

Detection agent antibodies may bind or interact with any number of locations on or along an allergen protein. Allergen antibody target sites contemplated include any and all possible sites for detecting particular allergens.

Detection agent compounds of the present invention may be selected for their ability to bind (reversibly or irreversibly) to one or more epitopes on a specific target allergen. Epitopes on target allergens may include, but are not limited to, one or more feature, region, domain, chemical group, functional group, or moiety. Such epitopes may be made up of one or more atom, group of atoms, atomic structure, molecular structure, cyclic structure, hydrophobic structure, hydrophilic structure, sugar, lipid, amino acid, peptide, glycopeptide, nucleic acid molecule, or any other antigen structure.

Detection agent antibodies of the present invention are primarily amino acid-based molecules. These molecules may be “peptides,” “polypeptides,” or “proteins.” In some cases, antibodies may include non amino acid-based molecules.

### Antibody Conjugates

In some embodiments, the antibodies of the present invention may be conjugated with one or more detectable label for purposes of detection according to methods known in the art. The label can be a radioisotope, fluorescent compound, chemiluminescent compound, enzyme, or enzyme co-factor, or any other labels known in the art. In some aspects, the antibody that binds to a desired target (also referred to herein as a “primary antibody”) is not labeled, but may be detected by binding of a second antibody that specifically binds to the primary antibody (referred to herein as a “secondary antibody”). According to such methods, the secondary antibody may include a detectable labeled.

In some embodiments, enzymes that can be attached to antibodies may include, but are not limited to horseradish peroxidase (HRP), alkaline phosphatase, and glucose Oxidase (GOx). Fluorescent compounds may include, but are not limited to, ethidium bromide; fluorescein and derivatives thereof (e.g., FITC); cyanine and derivatives thereof (e.g. indocarbocyanine, oxacarbocyanine, thiacarbocyanine, and merocyanine); rhodamine; oregon green; eosin; texas red; nile red; nile blue; cresyl violet; oxazine 170; proflavin; acridine orange; acridine yellow; auramine; crystal violet; malachite green; porphin; phthalocyanine; bilirubin; allophycocyanin (APC); green fluorescent protein (GFP) and variants thereof (e.g., yellow fluorescent protein YFP, blue fluorescent protein BFP, and cyan fluorescent protein CFP); ALEXIFLOUR® compounds (Thermo Fisher Scientific, Waltham, Mass.); and quantum dots. Other conjugates that may be used to label antibodies may include biotin, avidin, and streptavidin.

Antibody labels may be used to generate a detection signal (e.g., fluorescent, colorimetric, luminescent, enzymatic, radioactive, etc.) that can be detected to indicate the presence or absence of an allergen in a test sample.

### Variations

Amino acid-based detection agents or target allergens of the invention may exist as a whole polypeptide, a plurality of polypeptides or fragments of polypeptides, which independently may be encoded by one or more nucleic acids, a plurality of nucleic acids, nucleic acid fragments or variants of any of the aforementioned. As used herein, the term “polypeptide” refers to a polymer of amino acid residues (natural or unnatural) linked together most often by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

As used herein, the term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Variants may possess at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5% or at least about 99.9% amino acid sequence identity (homology) to a native or reference sequence.

In some embodiments “variant mimics” are provided. As used herein, the term “variant mimic” refers to a variant which contains one or more amino acids which would mimic an activated sequence. For example, glutamate may serve as a mimic for phospho-threonine and/or phospho-serine. Alternatively, variant mimics may result in deactivation or in an inactivated product containing the mimic, e.g., phenylalanine may act as an inactivating substitution for tyrosine; or alanine may act as an inactivating substitution for serine. The amino acid sequences of the compounds and/or compositions of the invention may comprise naturally occurring amino acids and as such may be considered to be proteins, peptides, polypeptides, or fragments thereof. Alternatively, the compounds and/or compositions may comprise both naturally and non-naturally occurring amino acids.

As used herein, the term “amino acid sequence variant” refers to molecules with some differences in their amino acid sequences as compared to a native or starting sequence. The amino acid sequence variants may possess substitutions, deletions, and/or insertions at certain positions within the amino acid sequence. As used herein, the terms “native” or “starting” when referring to sequences are relative terms referring to an original molecule against which a comparison may be made. Native or starting sequences should not be confused with wild type sequences. Native sequences or molecules may represent the wild-type (that sequence found in nature) but do not have to be identical to the wild-type sequence.

Ordinarily, variants will possess at least 50%, at least 60%, at least 70%, at least 75%, at least 80%, at least 85%, at least 90%, at least 95%, at least 98%, at least 99% or at least 99.5% amino acid sequence identity in comparison to a native sequence.

As used herein, the term “identity” as is applies to amino acid sequences is defined as the percentage of residues in the candidate amino acid sequence that are identical with the residues in the amino acid sequence of a second sequence after aligning the sequences and introducing gaps, if necessary, to achieve the maximum percent identity. Methods and computer programs for alignment are well known in the art. It is understood that homology depends on a calculation of percent identity but may differ in value due to gaps and penalties introduced in the calculation.

As used herein, the term “homolog” as it applies to amino acid sequences is meant the corresponding sequence of other species having substantial identity to a second sequence of a second species.

As used herein, the term “analog” is meant to include polypeptide variants which differ by one or more amino acid alterations, e.g., substitutions, additions or deletions of amino acid residues that still maintain the properties of the parent polypeptide.

As used herein, the term “derivative” is used synonymously with the term “variant” and refers to a molecule that has been modified or changed in any way relative to a reference molecule or starting molecule.

The present invention contemplates several types of compounds and/or compositions which are amino acid based including variants and derivatives. These include substitutional, insertional, deletional and covalent variants and derivatives. As such, included within the scope of this invention are compounds and/or compositions comprising substitutions, insertions, additions, deletions and/or covalent modifications.

Amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein, the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

As used herein, the term “insertional variants” when referring to proteins are those with one or more amino acids inserted immediately adjacent to an amino acid at a particular position in a native or starting sequence. As used herein, the term “immediately adjacent” refers to an adjacent amino acid that is connected to either the alpha-carboxy or alpha-amino functional group of a starting or reference amino acid.

As used herein, the term “deletional variants” when referring to proteins, are those with one or more amino acids in the native or starting amino acid sequence removed. Ordinarily, deletional variants will have one or more amino acids deleted in a particular region of the molecule.

As used herein, the term “derivatives,” as referred to herein includes variants of a native or starting protein comprising one or more modifications with organic proteinaceous or non-proteinaceous derivatizing agents, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337.

As used herein, the term “features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein, the term “surface manifestation” when referring to proteins refers to a polypeptide based component of a protein appearing on an outermost surface.

As used herein, the term “local conformational shape” when referring to proteins refers to a polypeptide based structural manifestation of a protein which is located within a definable space of the protein.

As used herein, the term “fold”, when referring to proteins, refers to the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein, the term “turn” as it relates to protein conformation, refers to a bend which alters the direction of the backbone of a peptide or polypeptide and may involve one, two, three or more amino acid residues.

As used herein, the term “loop,” when referring to proteins, refers to a structural feature of a peptide or polypeptide which reverses the direction of the backbone of a peptide or polypeptide and comprises four or more amino acid residues. Oliva et al. have identified at least 5 classes of protein loops (Oliva, B. et al., An automated classification of the structure of protein loops. J Mol Biol. 1997. 266(4):814-30).

As used herein, the term “half-loop,” when referring to proteins, refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein, the term “domain,” when referring to proteins, refers to a motif of a polypeptide having one or more identifiable structural or functional characteristics or properties (e.g., binding capacity, serving as a site for protein-protein interactions).

As used herein, the term “half-domain,” when referring to proteins, refers to a portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein, the terms “site,” as it pertains to amino acid based embodiments is used synonymously with “amino acid residue” and “amino acid side chain”. A site represents a position within a peptide or polypeptide that may be modified, manipulated, altered, derivatized or varied within the amino acid based molecules of the present invention.

As used herein, the terms “termini” or “terminus,” when referring to proteins refers to an extremity of a peptide or polypeptide. Such extremities are not limited only to the first or final site of the peptide or polypeptide but may include additional amino acids in the terminal regions. The polypeptide based molecules of the present invention may be characterized as having both an N-terminus [terminated by an amino acid with a free amino group (NH2)] and a C-terminus [terminated by an amino acid with a free carboxyl group (COOH)]. Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would. Modifications and manipulations can be accomplished by methods known in the art such as site directed mutagenesis.

In some embodiments, compounds and/or compositions of the present invention may comprise one or more atoms that are isotopes. As used herein, the term “isotope” refers to a chemical element that has one or more additional neutrons. In some embodiments, compounds of the present invention may be deuterated. As used herein, the term “deuterate” refers to the process of replacing one or more hydrogen atoms in a substance with deuterium isotopes. Deuterium isotopes are isotopes of hydrogen. The nucleus of hydrogen contains one proton while deuterium nuclei contain both a proton and a neutron. The compounds and/or compositions of the present invention may be deuterated in order to change one or more physical property, such as stability, or to allow compounds and/or compositions to be used in diagnostic and/or experimental applications.

### Antibodies Against Food Allergens

In some embodiments, antibodies used as detection agents may be single strained scFv antibodies that crossly react with major fish allergens, parvalbumins, from different fishes such as cod (Gad m 1), carp (Cyp c 1) and rainbow trout (Onc m 1) (Bublin et al., PloS One, 2015, 10(11): e0142625). ScFv antibodies against cockroach allergens obtained by screening libraries derived from naive human lymphocytes and hyperimmunized chicken splenocytes and bone marrow, may also be used as detection agents in assays for determining cockroach allergens (Khurana et al., PLoS One, 2015, 10(10), e0140225). The antibodies may be monoclonal antibodies specific to soybean glycinin such as Mab 3B2 and 4B2 developed by Ma et al. (Ma et al., Food Chemistry, 2010, 121(2): 546-551); recombinant antibodies from phage display libraries that bind apple allergen protein Mal d1 and celery allergen protein Api g1 (Haka et al., Clinical and Translational Allergy 2015, 5 (Suppl 3):P52); monoclonal antibody E58 that binds to Jun a 1, the group 1 allergen of the mountain cedar (Juniperus ashei, Cupressaceae) (Goldblum et al., Mol Immunol., 2016, 74: 106-112); and monoclonal antibodies against Der f2, one of the major allergens of the house dust mite Dermatophagoides farina (Chen et al., Drug Discov. Ther., 2016, 10(2): 103-108). In other embodiments, antibodies may be commercial antibodies available for food allergen detection. These antibodies may be polyclonal antibodies, monoclonal antibodies, and/or recombinant antibodies. As non-limiting examples, antibodies may include polyclonal anti-ovalbumin (Anti-OVA) antibodies from Olympus (Olympus America Inc., PA USA), anti-major latex allergen Hev b5 antibody from Abcam (Abcam, USA), monoclonal antibodies specific to Cry j1 and Cry j2, the cedar (Cryptomeria japonica) pollen allergen, Anti ovomucoid monoclonal antibody, Anti casein monoclonal antibody, Anti casectoglobulin monoclonal antibody, Anti gliadin monoclonal antibody and Anti Ascaris polyclonal antibody (COSMO BIO, JP)

### Targets: Allergens

In accordance with the present invention, detection methods may be used to detect any allergen in a sample. The allergen may include, but is not limited to, a food allergen, an allergen from the environment such as plants, animals, microorganisms, air or water, and a medical allergen (i.e., any allergen found in a medicine or medical device). In some embodiments, allergens are food allergens. Examples of allergenic proteins associated with food include, but are not limited to, allergens associate with Brine shrimp (Art fr 5), Crab (Cha f 1), North Sea Shrimp (Cra c 1, Cra c 2, Cra c 4, Cra c 5, Cra c 6, Cra c 8), American lobster (Hom a 1, Hom a 3, Hom a 6), white shrimp (Lit v 1, Lit v 2, Lit v 3, Lit v4), giant freshwater prawn (Mac r 1), shrimp (Met e 1, Pen a 1, Pen i 1), northern shrimp (Pan b 1), spiny lobster (Pan s 1), black tiger shrimp (Pen m 1, Pen m 2, Pen m 3, Pen m 4, Pen m 6), narrow-clawed crayfish (Pon i 4, Pon i 7), blue swimmer crab (Por p 1), domestic cattle (Bos d 4, Bos d 5, Bos d 6, Bos d 7, Bos d 8, Bos d 9, Bos d 10, Bos d 11, Bos d 12), Atlantic herring (Clu h 1), common carp (Cyp c 1), Baltic cod (Gad c 1), Atlantic cod (Gad m 1, Gad m 2, Gad m 3), cod (Gad c 1), chicken (Gal d 1, Gal d 2, Gal d 3, Gal d 4, Gal d 5), Barramunda (Lat c 1), Lepidorhombus whiffiagonis (Lep w 1), chum salmon (Onc k 5), Atlantic salmon (Sal s 1, Sal s 2, Sal s 3) rainbow trout (Onc m 1), Mozambique tilapia (Ore m 4), edible frog (Ran e 1, Ran e 2), pacific pilchard (Sar sa 1), ocean perch (Seb m 1), yellowfin tuna (Thu a 1, Thu a 2, Thu a 3), swordfish (Xip g 1), abalone (Hal m 1), brown garden snail (Hel as 1), Squid (Tod p 1), pineapple (Ana c 1, Ana c 2), asparagus (Aspa o 1), barley (Hor v 12, Hor v 15, Hor v 16, Hor v 17, Hor v 20, Hor v 21), banana (Mus a 1, Mus a 2, Mus a 3, Mus a 4, Mus a 5), banana (Musxpl), rice (Ory s 12), rye (Sec c 20), wheat (Tri a 12, Tri a 14, Tri a 18, Tri a 19, Tri a 25, Tri a 26, Tri a 36, Tri a 37), maize (corn) (Zea m 14, Zea m 25), kiwi fruit (Act c1, Act c 2, Act c 5, Act c 8, Act c 10, Act d 1, Act d 2, Act d 3, Act d 4, Act d 5, Act d 6, Act d 7, Act d 8, Act d 9, Act d 10, Act d 11), cashew (Ana o 1, Ana o 2, Ana o 3), celery (Api g 1, Api g 2, Api g 3, Api g 4, Api g 5, Api g 6), peanut (Ara h 1, Ara h 2, Ara h 3, Ara h 4, Ara h 5, Ara h 6, Ara h 7, Ara h 8, Ara h 9, Ara h 10, Ara h 11, Ara h 12, Ara h 13), brazil nut (Ber e 1, Ber e 2), oriental mustard (Bra j 1), rapeseed (Bra n 1), cabbage (Bra o 3), turnip (Bra r 1, Bra r 2), bell pepper (Cap a 1w, Cap a 2), pecan (Car i 1, Car i 2, Car i 4), chestnut (Cas s 1, Cas s 5, Cas s 8, Cas s 9), lemon (Cit I 3), tangerine (Cit r 3), sweet orange (Cit s 1, Cit s 2, Cit s 3), Hazel (Cor a 1, Cor a 2, Cor a 8, Cor a 9, Cor a 11, Cor a 12, Cor a 13, Cor a 14), muskmelon (Cuc m 1, Cuc m 2, Cuc m 3), carrot (Dau c 1, Dau c 4, Dau c 5), common buckwheat (Fag e 2, Fag e 3), tartarian buckwheat (Fag t 2), strawberry (Fra a 1, Fra a 3, Fra a 4), soybean (Gly m 1, Gly m 2, Gly m 3, Gly m 4, Gly m 5, Gly m 6, Gly m 7, Gly m 8), sunflower (Hel a1, Hel a 2, Hel a 3), black walnut (Jug n 1, Jug n 2), English walnut (Jug r 1, Jug r 2, Jug r 3, Jug r 4), Cultivated lettuce (Lac s 1), Lentil (Len c 1, Len c 2, Len c 3), litchi (Lit c 1), narrow-leaved blue lupin (Lup an 1), apple (Mal d 1, Mal d 2, Mal d 3, Mal d 4), Cassava (Man e 5), mulberry (Morn 3), avocado (Pers a 1), green bean (Pha v 3), pistachio (Pis v 1, Pis v 2, Pis v 3, Pis v 4, Pis v 5), pea (Pis s 1, Pis s 2), apricot (Pru ar 1, Pru ar 3), sweet cherry (Pru av 1, Pru av 2, Pru av 3, Pru av 4), European plum (Pru d 3), almond (Pru du 3, Pru du 4, Pru du 5, Pru du 6), peach (Pru p 1, Pru p 2, Pru p 3, Pru p 4, Pru p 7), pomegranate (Pun g 1), pear (Pyr c 1, Pyr c 3, Pyr c 4, Pyr c 5), castor bean (Ric c 1), red raspberry (Rub i 1, Rub i 3), Sesame (Ses i 1, Ses i 2, Ses i 3, Ses i 4, Ses i 5, Ses i 6, Ses i 7), yellow mustard (Sin a 1, Sin a 2, Sin a 3, Sin a 4), tomato (Sola I 1, Sola I 2, Sola I 3, Sola I 4), potato (Sola t 1, Sola t 2, Sola t 3, Sola t 4), Mung bean (Vig r 1, Vig r 2, Vig r 3, Vig r 4, Vig r 5, Vig r 6), grape (Vit v 1), Chinese date (Ziz m 1), Anacardium occidentale (Ana o 1.0101, Ana o 1.0102), Apium graveolens (Api g 1.0101, Api g 1.0201), Daucus carota (Dau c1.0101, Dau c1.0102, Dau c1.0103, Dau c1.0104, Dau c1.0105, Dau c1.0201), Citrus sinensis (Cit s3.0101, Cit s3.0102), Glycine max (Gly m1.0101, Gly m1.0102, Gly m3.0101, Gly m3.0102), Lens culinaris (Len c1.0101, Len c1.0102, Len c1.0103), Pisum sativum (Pis s1.0101, Pis s1.0102), Lycopersicon sativum (Lyc e2.0101, Lyc e2.0102), Fragaria ananassa (Fra a3.0101, Fra a3.0102, Fra a3.0201, Fra a3.0202, Fra a3.0203, Fra a3.0204, Fra a3.0301), Malus domestica (Mal d1.0101, Mal d1.0102, Mal d1.0103, Mal d1.0104, Mal d1.0105, Mal d1.0106, Mal d1.0107, Mal d1.0108, Mal d1.0109, Mal d1.0201, Mal d1.0202, Mal d1.0203, Mal d1.0204, Mal d1.0205, Mal d1.0206, Mal d1.0207, Mal d1.0208, Mal d1.0301, Mal d1.0302, Mal d1.0303, Mal d1.0304, Mal d1.0401, Mal d1.0402, Mal d1.0403, Mal d3.0101w, Mal d3.0102w, Mal d3.0201w, Mal d3.0202w, Mal d3.0203w, Mal d4.0101, Mal d4.0102, Mal d4.0201, Mal d4.0202, Mal d4.0301, Mal d4.0302), Prunus avium (Pru av1.0101, Pru av1.0201, Pru av1.0202, Pru av1.0203), and Prunus persica (Pru p4.0101, Pru p4.0201); and any variants thereof. The names of allergens associated with food are systematically named and listed according to IUIS Allergen Nomenclature Sub-Committee (see, International Union of Immunological Societies Allergen Nomenclature Sub-Committee, List of isoallergens and variants.)

In addition to food allergens, methods of the present invention may detect airborne particulates/allergens and other environmental allergens. Samples that contain allergens may be obtained from plants (e.g. weeds, grasses, trees, pollens), animals (e.g., allergens found in the dander, urine, saliva, blood or other bodily fluid of mammals such as cat, dog, cow, pig, sheep, horse, rabbit, rat, guinea pig, mouse and gerbil), fungi/mold, insects (e.g., stinging insects such as bee, wasp, and hornet and chirnomidae (non-biting midges), as well as other insects such as the housefly, fruit fly, sheep blow fly, screw worm fly, grain weevil, silkworm, honeybee, non-biting midge larvae, bee moth larvae, mealworm, cockroach and larvae of Tenibrio molitor beetle; spiders and mites such as the house dust mite), rubbers (e.g. latex), metals, chemicals (e.g. drugs, protein detergent additives) and autoallergens and human autoallergens (e.g. Hom s 1, Hom s 2, Hom s 3, Hom s 4, Hom s 5) (see, Allergen Nomenclature: International Union of Immunological Societies Allergen Nomenclature Sub-Committee, List of allergens and Allergen Nomenclature: International Union of Immunological Societies Allergen Nomenclature Sub-Committee, List of isoallergens and variants).

Examples of allergenic proteins from plants that can be detected using methods of the present invention include, but are not limited to, ash (Fra e 1), Japanese cypress (Cha ol, Chao 2), sugi (Cry j1, Cry j 2), cypress (Cup a 1), common cypress (Cups 1, Cups 3), mountain cedar (Jun a 1, Jun a 2, Jun a 3, Jun s 1), prickly juniper (Jun o 4), eastern red cedar (Jun v 1, Jun v 3), sweet vernal grass (Ant o 1), saffron crocus (Cro s 1, Cro s 2), Bermuda grass (Cyn d 1, Cyn d 7, Cyn d 12, Cyn d 15, Cyn d 22w, Cyn d 23, Cyn d 24), orchard grass (Dac g 1, Dac g 2, Dac g 3, Dac g 4, Dac g 5), meadow fescue (Fes p 4), velvet grass (Hol I 1, Hol I 5), barley (Hor v 1, Hor v 5), rye grass (Lol p 1, Lol p 2, Lol p 3, Lol p 4, Lol p 11), bahia grass (Pas n 1), canary grass (Pha a 1, Pha a 5), timothy (Phl p 1, Phl p 2, Phl p 4, Phl p 5, Phl p 6, Phl p 7, Phl p 11, Phl p 12, Phl p 13), date palm (Pho d 2), Kentucky blue grass (Poa p 1, Poa p 5), rye (Sec c 1, Sec c 5, Sec c 38), Johnson grass (Sor h 1), wheat (Tri a 15, Tri a 21, Tri a 27, Tri a 28, Tri a 29, Tri a 30, Tri a 31, Tri a 32, Tri a 33, Tri a 34, Tri a 35, Tri a 39), maize (Zea m 1, Zea m 12), alder (Aln g 1, Aln g 4), redroot pigweed (Ama r 2), short ragweed (Amb a 1, Amb a 2, Amb a 3, Amb a 4, Amb a 5, Amb a 6, Amb a 7, Amb a 8, Amb a 9, Amb a 10, Amb a 11), western ragweed (Amb p 5), giant ragweed (Amb t 5), mugwort (Art v 1, Art v 2, Art v 3, Art v 4, Art v 5, Art v 6), sugar beet (Beta v 1, beta v 2), European white birch (Bet v 1, Bet v 2, Bet v 3, Bet v 4, Bet v 6, Bet v 7), turnip (Bra r 5), hornbeam (Car b 1), chestnut (Cas s 1), rosy periwinkle (Cat r 1), lamb's-quarters, pigweed (Che a 1, Che a 2, Che a 3), Arabian coffee (Cof a 1, Cof a 2, Cof a 3), Hazel (Cor a 6, Cor a 10), Hazel nut (Cor a1.04, Cor a2, Cor a8), European beech (Fag s 1), ash (Fra e 1), sunflower (Hel a 1, Hel a 2), para rubber tree (Hey b 1, Hev b 2, Hev b 3, Hey b 4, Hev b 5, Hev b 6, Hev b 7, Hev b 8, Hev b 9, Hev b 10, Hev b 11, Hev b 12, Hev b 13, Hey b 14), Japanese hop (Hum j 1), privet (Lig v 1), Mercurialis annua (Mer a 1), olive (Ole e 1, Ole e 2, Ole e 3, Ole e 4, Ole e 5, Ole e 6, Ole e 7, Ole e 8, Ole e 9, Ole e 10, Ole e 11), European hophornbeam (Ost c 1), Parietaria judaica (Par j 1, Par j 2, Par j 3, Par j 4), Parietaria officinalis (Par o 1), Plantago lanceolata (Pal I 1), London plane tree (Pla a 1, Pla a 2, Pla a 3), Platanus orientalis (Pla or 1, Pla or 2, Pla or 3), white oak (Que a 1), Russian thistle (Sal k 1, Sal k 2, Sal k 3, Sal k 4, Sal k 5), tomato (Sola I 5), Lilac (Syr v 1, Syr v 5), Russian-thistle (Sal k 1), English plantain (Pla 11), Ambrosia artemisiifolia (Amb a8.0101, Amb a8.0102, Amb a9.0101, Amb a9.0102), Plantago lanceolata (Pla l1.0101, Pla l1.0102, Pla l1.0103), Parietaria judaica (Par j 3.0102), Cynodon dactylon (Cyn d1.0101, Cyn d1.0102, Cyn d1.0103, Cyn d1.0104, Cyn d1.0105, Cyn d1.0106, Cyn d1.0107, Cyn d1.0201, Cyn d1.0202, Cyn d1.0203, Cyn d1.0204), Holcus lanatus (Hol I1.0101, Hol I1.0102), Lolium perenne (Phl p1.0101, Phl p1.0102, Phl p4.0101, Phl p4.0201, Phl p5.0101, Phl p5.0102, Phl p5.0103, Phl p5.0104, Phl p5.0105, Phl p5.0106, Phl p5.0107, Phl p5.0108, Phl p5.0201, Phl p5.0202), Secale cereale (Sec c20.0101, Sec c20.0201), Betula Verrucosa (Bet v1.0101, Bet v1.0102, Bet v 1.0103, Bet v 1.0201, Bet v 1.0301, Bet v1.0401, Bet v 1.0402, Bet v 1.0501, Bet v 1.0601, Bet v 1.0602, Bet v1.0701, Bet v1.0801, Bet v1.0901, Bet v1.1001, Bet v1.1101, Bet v1.1201, Bet v 1.1301, Bet v1.1401, Bet v1.1402, Bet v1.1501, Bet v1.1502, Bet v1.1601, Bet v1.1701, Bet v 1.1801, Bet v1.1901, Bet v1.2001, Bet v1.2101, Bet v1.2201, Bet v1.2301, Bet v1.2401, Bet v 1.2501, Bet v1.2601, Bet v1.2701, Bet v1.2801, Bet v1.2901, Bet v1.3001, Bet v1.3101, Bet v 6.0101, Bet v6.0102), Carpinus betulus (Car b1.0101, Car b1.0102, Car b1.0103, Car b1.0104, Car b1.0105, Car b1.0106, Car b1.0106, Car b1.0106, Car b1.0106, Car b1.0107, Car b1.0107, Car b1.0108, Car b1.0201, Car b1.0301, Car b1.0302), Corylus avellana (Cor a1.0101, Cor a1.0102, Cor a1.0103, Cor a1.0104, Cor a1.0201, Cor a1.0301, Cor a1.0401, Cor a1.0402, Cor a1.0403, Cor a1.0404), Ligustrum vulgare (Syr v1.0101, Syr v1.0102, Syr v1.0103), Ligustrum lucidum pollen proteins (Profilin, Enolase, Fra e 9.01 (β-1,3-glucanase), Pollen-specific Polygalacturonases, Alanine aminotransferase and ATP synthease beta subunit) (Mani et al., Biochem Biophys Res Commun., 2015, 468(4): 788-792), Cryptomeria japonica (Cry j2.0101, Cry j2.0102), and Cupressus sempervirens (Cup s1.0101, Cup s1.0102, Cup s1.0103, Cup s1.0104, Cup s1.0105); and any variants thereof.

Lupin is an herbaceous plant of the leguminous family belonging to the genus Lupinus. In Europe, lupin flour and seeds are widely used in bread, cookies, pastry, pasta, sauces, as well as in beverages as a substitute for milk or soy, and in gluten-free foods. The International Union of Immunological Societies (IUIS) allergen nomenclature subcommittee recently designated β-conglutin as the Lup an 1 allergen. (Nadal, et al., (2012) DNA Aptamers against the Lup an 1 Food Allergen. PLoS ONE 7(4): e35253), and more recently, a high-affinity 11-mer DNA aptamer against Lup an 1 (β-conglutin) was reported (Nadal, et al., (2013) Probing high-affinity 11-mer DNA aptamer against Lup an 1 (β-conglutin). Anal. Bioanal. Chem. 405:9343-9349).

Examples of allergenic proteins from mites that can be detected using methods of the present invention include, but are not limited to, mite (Blo t 1, Blo t 3, Blo t 4, Blo t 5, Blot 6, Blot 10, Blot 11, Blot 12, Blo t 13, Blot 19, Blot t 21); American house dust mite (Der f 1, Der f 2, Der f 3, Der f 7, Der f 10, Der f 11, Der f 13, Der f 14, Der f 15, Der f 16, Der f 17, Der f 18, Der f 22, Der f 24); Dermatophagoides microceras (house dust mite) (Der m 1); European house dust mite (Der p 1, Der p 2, Der p 3, Der p 4, Der p 5, Der p 6, Der p 7, Der p 8, Der p 9, Der p 10, Der p 11, Der p 14, Der p 15, Der p 20, Der p 21, Der p 23); Euroglyphus maynei (House dust mite) (Eur m 2, Eur m 2, Eur m 3, Eur m 4, Eur m 14); storage mite (Aca s 13, Gly d 2, Lep d 2, Lep d 5, Lep d 7, Lep d 10, Lep d 13, Tyr p 2, Tyr p 3, Tyr p 10, Tyr p 13, Tyr p 24), Dermatophagoides farinae (Der f1.0101, Der f1.0102, Der f1.0103, Der f1.0104, Der f1.0105, Der f2.0101, Der f2.0102, Der f2.0103, Der f2.0104, Der f2.0105, Der f2.0106, Der f2.0107, Der f2.0108, Der f2.0109, Der f2.0110, Der f2.0111, Der f2.0112, Der f2.0113, Der f2.0114, Der f2.0115, Der f2.0116, Der f2.0117), Dermatophagoides pteronyssinus (Der p1.0101, Der p1.0102, Der p1.0103, Der p1.0104, Der p1.0105, Der p1.0106, Der p1.0107, Der p1.0108, Der p1.0109, Der p1.0110, Der p1.0111, Der p1.0112, Der p1.0113, Der p1.0114, Der p1.0115, Der p1.0116, Der p1.0117, Der p1.0118, Der p1.0119, Der p1.0120, Der p1.0121, Der p1.0122, Der p1.0123, Der p2.0101, Der p2.0102, Der p2.0103, Der p2.0104, Der p2.0105, Der p2.0106, Der p2.0107, Der p2.0108, Der p2.0109, Der p2.0110, Der p2.0111, Der p2.0112, Der p2.0113), Euroglyphus maynei (Eur m2.0101, Eur m2.0102), Lepidoglyphus destructor (Lep d2.0101, Lep d2.0101, Lep d2.0101, Lep d2.0102, Lep d2.0201, Lep d2.020) and Glycyphagus domesticus (Gly d2.0101, Gly d2.0201); and any variants thereof.

Examples of allergenic proteins from animals that can be detected using methods of the present invention include, but are not limited to, domestic cattle (Bos d 2, Bos d 3, Bos d 4, Bos d 5, Bos d 6, Bos d 7, Bos d 8), dog (Can f 1, Can f 2, Can f 3, Can f 4, Can f 5, Can f 6), domestic horse (Equ c 1, Equ c 2, Equ c 3, Equ c 4, Equ c 5), cat (Fel d 1, Fel d 2, Fel d 3, Fel d 4, Fel d 5w, Fel d 6w, Fel d 7, Fel d 8), mouse (Mus m 1), guinea pig (Cav p 1, Cav p 2, Cav p 3, Cav p 4, Cav p 6), rabbit (Ory c 1, Ory c 3, Ory c 4) rat (Rat n 1), Bos domesticus (Bos d 2.0101, Bos d 2.0102, Bos d 2.0103) and Equus caballus (Equ c2.0101, Equ c 2.0102); and any variants thereof

Examples of allergenic proteins from insects that can be detected using methods of the present invention include, but are not limited to, yellow fever mosquito (Aed a 1, Aed a 2, Aed a 3), Eastern hive bee (Api c 1), giant honeybee (Api d 1), honey bee (Api m 1, Api m 2, Api m 3, Api m 4, Api m 5, Api m 6, Api m 7, Api m 8, Api m 9, Api m 10, Api m 11, Api m 12), pigeon tick (Arg r 1), German cockroach (Bla g 1, Bla g 2, Bla g 3, Bla g 4, Bla g 5, Bla g 6, Bla g 7, Bla g 8, Bla g 11), bumble bee (Bom p 1, Bom p 4, Bom t 1, Bom t 4), silk moth (Bomb m 1), midge (Chi k 10, Chi t 1, Chi t 1.01, Chi t 2, Chi t 2.0101, Chi t 2.0102, Chit 3, Chit 4, Chit 5, Chit 6, Chit 6.01, Chit 7, Chit 8, Chit 9), cat flea (Cte f 1, Cte f 2, Cte f 3), yellow hornet (Dol a 5), white face hornet (Dol m 1, Dol m 2, Dol m 5), biting midge (Fort 1, Fort 2), Savannah Tsetse fly (Glo m 5), Asian ladybeetle (Har a 1, Har a 2), silverfish (Lep s 1), booklouse (Lip b 1), Australian jumper ant (Myr p 1, Myr p 2, Myr p 3), American cockroach (Per a 1, Per a 3, Per a 6, Per a 7, Per a 9, Per a 10), Indian meal moth (Plo i 1, Plo i 2), wasp (Pol a 1, Pol a 2, Pol a 5, Pole 1, Pol e 4, Pole 5, Pol f 5, Pol g 1, Pol g 5, Pol m 5, Poly p 1, Poly s 5, Ves vi 5), Mediterranean paper wasp (Pol d 1, Pol d 4, Pol d 5), tropical fire ant (Sol g 2, Sol g 3, Sol g 4), Solenopsis invicta (red imported fire ant) (Sol I 1, Sol I 2, Sol I 3, Sol I 4), black fire ant (Sol r 2, Sol r 3), Brazilian fire ant (Sol s 2, Sol s 3), horsefly (Tab y 1, Tab y 2, Tab y 5), pine processionary moth (Tha p 1, Tha p 2), California kissing bug (Tria p 1), European hornet (Vesp c 1, Vesp c 5), Vespa magnifica (hornet) (Vesp ma 2, Vesp ma 5), Vespa mandarinia (Giant asian hornet) (Vesp m1, Vesp m 5), yellow jacket (Ves f 5, Ves g 5, Ves m 1, Ves m 2, Ves m 5), Vespula germanica (yellow jacket) (Ves p 5), Vespula squamosa (Yellow jacket) (Ves s 1, Ve s s5), Vespula vulgaris (Yellow jacket) (Ves v 1, Ves v 2, Ves v 3, Ves v 4, Ves v 5, Ves v 6), Blattella germanica (Bla g 1.0101, Bla g 1.0102, Bla g 1.0103, Bla g 1.02, Bla g 6.0101, Bla g 6.0201, Bla g 6.0301), Periplaneta Americana (Per a1.0101, Per a1.0102, Per a1.0103, Per a1.0104, Per a1.02, Per a3.01, Per a3.0201, Per a3.0202, Per a3.0203, Per a7.0101, Per a7.0102), Vespa crabo (Ves pc 5.0101, Ves pc 5.0101), Vespa mandarina (Vesp m 1.01, Vesp m 1.02); and any variants thereof.

Examples of allergenic proteins from fungi/mold that can be detected using methods of the present invention include, but are not limited to, Alternaria alternata (Alternaria rot fungus) (Alt a 1, Alt a 3, Alt a 4, Alt a 5, Alt a 6, Alt a 7, Alt a 8, Alt a 10, Alt a 12, Alt a 13), Aspergillus flavus (fungus) (Asp fl 13), Aspergillus fumigatus (fungus) (Asp f 1, Asp f2, Asp f3, Asp f4, Asp f5, Asp f6, Asp f7, Asp f 8, Asp f 9, Asp f 10, Asp f 11, Asp f 12, Asp f 13, Asp f 15, Asp f 16, Asp f 17, Asp f 18, Asp f 22, Asp f 23, Asp f 27, Asp f 28, Asp f 29, Asp f 34), Aspergillus niger (Asp n 14, Asp n 18, Asp n 25), Aspergillus oryzae (Asp o 13, Asp o 21), Aspergillus versicolor (Asp v 13), Candida albicans (Yeast) (Cand a 1, Cand a 3), Candida boidinii (Yeast) (Cand b 2), Cladosporium cladosporioides (Cla c 9, Cla c 14), Cladosporium herbarum (Cla h 2, Cla h 5, Cla h 6, Cla h 7, Cla h 8, Cla h 9, Cla h 10, Cla h 12), Curvularia lunata (Synonym: Cochliobolus lunatus) (Cur l 1, Cur I 2, Cur I 3, Cur I 4), Epicoccum purpurascens (Soil fungus) (Epi p 1), Fusarium culmorum (N.A.) (Fus c 1, Fus c 2), Fusarium proliferatum (Fus p 4), Penicillium brevicompactum (Pen b 13, Pen b 26), Penicillium chrysogenum (Pen ch 13, Pen ch 18, Pen ch 20, Pen ch 31, Pen ch 33, Pen ch 35), Penicillium citrinum (Pen c 3, Pen c 13, Pen c 19, Pen c 22, Pen c 24, Pen c 30, Pen c 32), Penicillium crustosum (Pen cr 26), Penicillium oxalicum (Pen o 18), Stachybotrys chartarum (Sta c 3), Trichophyton rubrum (Tri r 2, Tri r 4), Trichophyton tonsurans (Tri t 1, Tri t 4), Psilocybe cubensis (Psi c 1, Psi c 2), Shaggy cap (Cop c 1, Cop c 2, Cop c 3, Cop c 5, Cop c 7), Rhodotorula mucilaginosa (Rho m 1, Rho m 2), Malassezia furfur (Malaf2, Malaf3, Malaf4), Malassezia sympodialis (Malas1, Malas5, Malas6, Malas7, Malas8, Malas9, Malas10, Malas11, Malas12, Malas13) and Alternaria alternate (Alt a1.0101, Alt a1.0102); and any variants thereof.

Examples of additional allergens include, but are not limited to, Nematode (Ani s 1, Ani s 2, Ani s 3, Ani s 4), worm (Asc s 1), silkworm pupa (arginine kinase, glycoprotein, Jeong et al., J Korean Med, Sci., 2016, 31(1), 18-24), soft coral (Den n 1), rubber (Latex) (Hey b 1, Hev b 2, Hev b 3, Hev b 5, Hev b 6, Hev b 7, Hev b 8, Hev b 9, Hev b 10, Hev b 11, Hey b 12, Hev b 13), obeche (Trip s 1) and Heveabrasiliensis (Hey b6.01, Hev b6.0201, Hey b6.0202, Hev b6.03, Hev b8.0101, Hev b8.0102, Hev b8.0201, Hev b8.0202, Hev b8.0203, Hey b8.0204, Hev b10.0101, Hev b10.0102, Hev b10.0103, Hev b11.0101, Hev b11.0102); and any variants thereof.

In some embodiments, methods of the present invention may be used in a hospital for clinical food or medicine allergy test and to identify food/allergen(s) to which a patient is allergic. In addition, assays and methods of the present invention may be used as a carry-on tester for people who have food/environmental allergy, for example at home to test commercial food, or at restaurant to check dishes they ordered. The food sample could be fresh food, frozen food, cooked food or processed food containing animal derived meat and/or vegetables.

### Targets: Other Molecules

In some embodiments, methods of the present invention may be used to detect other target molecules, including but not limited to, pathogens from a pathogenic microorganism in a sample, such as bacteria, yeasts, fungi, spores, viruses or prions; disease proteins (e.g., biomarkers for diseases diagnosis and prognosis); pesticides and fertilizers remained in the environment; and toxins. In other embodiments, methods of the present invention may be used to detect non-protein targets such as minerals and small molecules (e.g., antibiotics).

### Detection Methods

### Antibody-Based Detection

Antibody-based methods typically involve the detection of allergens through binding of antibodies to the allergen and then detecting bound antibodies using one or more methods described herein below.

Many antibody-based methods are available for detection of allergenic proteins from an allergic source. Different formats include, but are not limited to, one-step antibody tests, two-step antibody tests, dot blot assays, reverse dot blot assays, sandwich-type assays, competitive binding assays, enzyme-linked immunosorbent assays (ELISAs), and strip tests.

Many ELISAs and strip tests for detection of different allergens have been developed for use in the food industry as well as for individual use (e.g., through the use of portable detection devices, see US Patent Publication NOs.: 2014/0295406 and 2015/0011020, the contents of each of which are herein incorporated by reference in their entirety). In some cases, strip tests involve the formation of complexes between anti-allergen antibody-coated beads on the test strip and allergens in a sample. When colored beads are used, these complexes give rise to a colored test line on the strip, indicating a positive (i.e., allergen-containing) sample. In a similar way, a colored control band is formed, indicating that the test has been carried out correctly. In many ELISA assays, allergenic compounds are detected by specific enzyme-labeled antibodies that may be visualized by enzymatic reaction leading to the formation of colored products. Such products may be analyzed using, for example, a spectrophotometer.

Antibody-based methods of the invention can detect levels of allergens in food products at concentrations in the low parts per million (ppm) ranges.

In some embodiments, assays of the invention involve contacting a substrate with a test sample and then detecting allergens bound to the substrate with allergen-specific antibodies. According to such assays, the allergen-specific primary antibody may be conjugated with a detectable label. In other cases, the primary antibody may be detected with a secondary antibody comprising a detectable label. In additional cases, the detectable label may be biotin that may subsequently be contacted with an avidin or streptavidin-based compound comprising a detectable label.

In some embodiments, assays of the invention involve providing a substrate with bound primary antibodies. When such substrates are contacted with a test sample, target allergens will bind to the primary antibody and may be detected with a second allergen-specific antibody that may be conjugated to a detectable label. In some cases, the detectable label may be biotin that may be detected by an additional avidin or streptavidin-based compound comprising a detectable label.

In some embodiments, assays of the invention involve providing a substrate with bound primary antibodies, wherein the substrate is a chip that can detect changes in bound versus unbound primary antibodies (e.g., as ini SPR-based assays).

Substrates used in antibody-based detection methods may include, but are not limited to, test strips, membranes, hydrophobic membranes, hydrophilic membranes, polymeric membranes, glass, glass plates, glass dishes, glass slides, plastic, plastic dishes, plastic plates, computer chips, cells, beads, tubes, and channels.

### Sensor-Based Detection

Recently, biosensor immunoassays, such as those involving the use of surface plasmon resonance (SPR)-based biosensors, have become increasingly accepted methods for allergen detection. According to such methods, specific detection agents may be immobilized on the surface of a chip and an SPR-based detection device measures changes in the refractive index at the surface of a sensor chip, caused by the binding of allergens to an immobilized detection agent. The binding of allergens in the sample is followed in real time, and from the change in the signal, the concentration in the sample can be calculated from a calibration curve.

### Test Samples

Devices and methods of the invention may be used to detect target allergens in test samples in a variety of formats. Test samples may be solid, liquid or gaseous and may be obtained from any number of sources, including, but not limited to, foods, soil, air, beverages, bodies of water, furniture, carpets, clothing, fur, pelts, creams, lotions, and medicines.

In some embodiments, a measured portion of a test sample is collected before the sample is processed for a detection assay, e.g. homogenization in a sample processing chamber or the like of a detection device. The recommended amount of a test sample may vary for different food products. In some aspects, a weighing mechanism may be provided when picking up a portion of a test sample to ensure an adequate amount of sample is collected. In other aspects, the collected sample may be pre-processed during the collection such as being cut into small pieces, or being smashed as small particles, or being heated up to a liquid state. Such pre-processing may facilitate further homogenization in a sample processing chamber or the like of a test container in a detection device and assist in sample digestion and efficient allergen extraction.

In some embodiments, an extraction buffer may be used to digest the collected sample and extract allergens. Such extraction buffers may be further optimized for universal use, or specialized for a particular type of sample, e.g., a cooked meat product. In some aspects, the extraction buffer may be optimized by choosing different buffering agents at different concentrations and pH values, lysis reagents, detergents, reducing agents, protease inhibitors, surfactants and/or other chemicals. Such extraction buffers, together with improved processing procedures can ensure efficient extraction of allergens from the test sample.

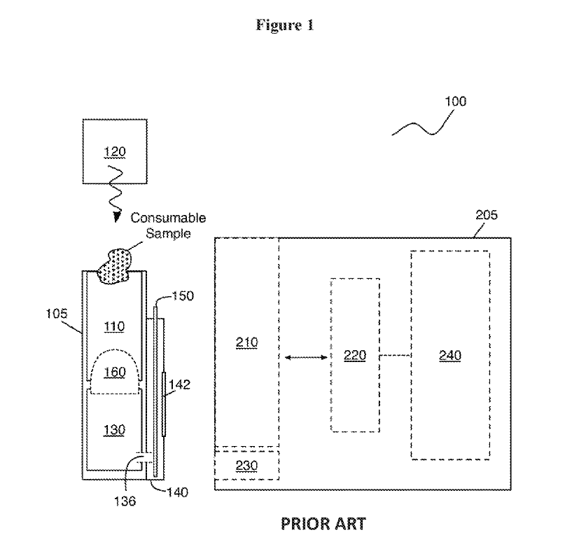

In some embodiments, the pre-processed test sample when added into an extraction buffer of the present invention may form slurry, a mix of all materials released from the test sample. This slurry may be further homogenized. As used herein, the term “homogenize” refers to a process whereby the components of a sample, blend, mixture, buffer, or the like are distributed in a uniform manner. In some cases, samples may be homogenized by a homogenizer provided in a detection device. In accordance with the present invention, the homogenizer may be provided with one or more bladders, grinders, blenders or a set of protrusions arranged in a uniform distribution at one end, and/or the combination thereof. In some aspects, the homogenizer is assembled in a food processing chamber in a detection device and controlled by a motor integrated in the detection device (e.g., the first chamber 110 of the portable test container 105 in the detection device 100 (FIG. 1) described by Sundvor et al. (U.S. Patent Publication NO.: 2015/0011020, which is incorporated by reference herein in its entirety).

In some aspects, the homogenizer may be further provided with a heating/cooling mechanism to control the temperature during the homogenization.

In certain embodiments, allergen isolation from the test sample may further comprise a procedure to separate allergen proteins, or total proteins from non-protein components (e.g., lipids, nucleic acids) or other debris.

In some embodiments, filtration may be used to further isolate allergens, before the extraction solution is processed to the next step, for example, being transmitted to a second chamber or the like of the test container or the like in a detection device. Filtration may include a membrane filter positioned between the sample processing chamber and the second chamber of the test container or the like in a detection device. Test sample filtering may lower the background signals generated during detection assay procedures, in particular in antibody binding assays.

Where amino acid-based allergens are detected, total proteins isolated from a test sample during a specific allergen assay may be measured. Such measurement can be used to monitor the efficiency of extraction and detection assay success. For example, if total protein levels are low in the extracted test sample, this finding may be used to indicate that lack of a detection signal represents a false negative result (due to lack of allergen proteins extracted from the test sample). Additionally, total proteins may be used to normalize the allergen concentration in a test sample. In some aspects, the total protein concentration may be determined using any available total protein determination assays such as Coomassie Blue assay, and Pyrogallol Red Protein Dye-Binding assay.

### Devices

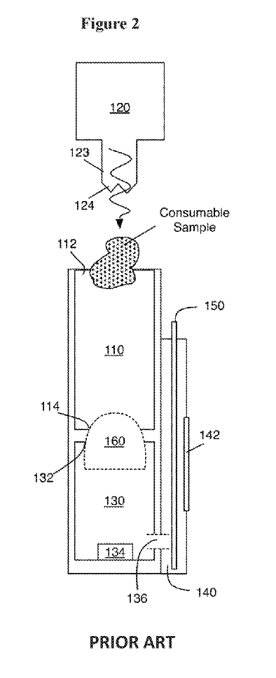

In some embodiments of the present invention, processed samples (i.e. extracted allergens) are transmitted to an analytical area within a portable test container (e.g., such as any of the test containers described in US Publication No. 2015/0011020, including, but not limited to, the test container labeled 105 in FIGS. 1 and 2 of that application), or the like, in a detection device for further analysis. In some aspects, detection agents (e.g., antibodies) that specifically recognize target allergens may be provided in the analytical area of the test container. The binding between extracted allergens and detection agents may be used to generate signals that can be detected and measured through a detection window within detection devices of the invention. Such signals will be analyzed and read as indicators that inform a user of the presence, absence and/or amount of an allergen tested in the test sample. The signals may be colorimetric changes, fluorescent signals, or any other signal type described herein.

In some embodiments, methods of the present invention include using the devices described in US Patent Publication NOs.: 2014/0295406 and 2015/0011020 (the contents of each of which are herein incorporated by reference in their entirety) in combination with any of the detection agents (e.g., antibodies) of the present invention to detect one or more target allergens in a test sample.

In some embodiments, devices of the present invention may be used to carry out methods for detecting allergens in test samples using the following steps: (a) obtaining a test sample; (b) receiving the test sample in a first chamber of a test container in a detection device through a reception port of the first chamber configured to receive the test sample; (c) processing the test sample in the first chamber by homogenization in an extraction buffer; (d) delivering the processed sample to a second chamber of the test container through a second port of the first chamber, wherein said second port is configured to deliver the processed sample in the first chamber to the second chamber, and wherein the second chamber is configured to receive the processed sample from the second port of the first chamber; (e) contacting the processed sample with one or more detection agent (capable of binding the allergen being detected); and (f) observing a detection signal.

### Detection Methods and Assays: Food Allergens

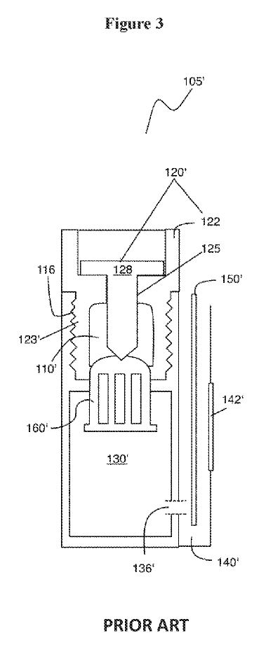

In accordance with the present invention, methods and assays optimized for detecting the presence, absence and/or amount of one or more allergens in a food sample may comprise the steps of i): collecting a measured portion of the food sample; ii): receiving the collected sample at a first chamber of a disposable test container or the like, through a reception port of the first chamber configured to receive the collected sample; iii): processing the collected sample in the first chamber through homogenization; iv): delivering the homogenized sample to a second chamber (e.g., the second chamber 130 described in US Publication No. 2015/0011020) (FIGS. 2 and 3) of the test container or the like through a second port of the first chamber which is configured to deliver the processed sample in the first chamber to the second chamber; v): extracting allergen proteins from the homogenized sample in an extraction buffer; and mixing the processed sample with one or more detection agents that specifically bind to the one or more allergens within an analytic chamber (e.g., the analysis chamber 140 described in US Publication No. 2015/0011020) (FIGS. 1 and 2) or the like in the test container; vi): detecting the interaction between allergens in the sample and detection agents through a detection window that enables the detection of signals from the interaction; and vii): indicating the presence, absence and/or amount of the one or more allergens in the tested sample.

In some embodiments, the detection agents used in the present invention are antibodies. The detection assays may be immunological assays such as ELISAs, strip tests, immunological biosensors, antibody-coated chips and other antibody based methods. In some aspects, an ELISA method may be a direct ELISA, a quantitative sandwich ELISA, or a competitive ELISA. The sandwich ELISA assay may be implemented in an analysis chamber of a detection device (i.e. the analysis chamber 140 of US Patent Publication NO.: 2015/0011020). A primary antibody (e.g., binding directly to the target allergen) against a target allergen may be used (directly labeled or in combination with a labeled secondary antibody) to measure a target allergen in a processed sample. In another embodiment, the detection assay may be a strip test wherein the antibody specifically binding to a target allergen may be bound to cellulose nanobeads and labeled with a colormetric agent or a fluorophore, as discussed above.

In some embodiments, antibodies may be provided in an analytic chamber of a detection device as a ready-to-use solution and mixed with a processed sample. In other embodiments, antibodies may be confined to a surface or substrate within an analytical chamber of a detection device (e.g., the detection substrate 150 of US Patent Publication NO.: 2015/0011020). The surface or substrate may be a test strip, a membrane, a membrane on a test strip, a plastic surface, a glass surface, a pad that can absorb and retain antibodies, a microwell surface, a biosensor chip surface, or any other solid surface types or substrates (including those described herein). In some cases, such surfaces may be pretreated with antibodies (or any other detection agents) before processed samples are provided. In some cases, the stored antibodies may be released into the analytic chamber and mixed with the processed sample during detection.

Any known antibodies that can detect specific allergens may be used according to the present methods. Either monoclonal or polyclonal antibodies may be used as detection agents.

In certain embodiments, one or more antibodies may be used in the immunological assays, depending on the nature of the food matrices. Some food contains several allergenic proteins (e.g., at least eight peanut proteins, such as Ara h1 and Ara h2), and can potentially cause an immunological response. In such cases, more than one antibody against more than one allergenic protein may be used in a mixed cocktail for detecting the presence, absence and/or amount of peanut in a sample. In other aspects, some food matrices such as fish, shellfish and mollusks, contain only one major allergenic protein. One or more antibodies that specifically bind to this major allergen protein may be used for allergen detection.

The ability of a detection assay and method in detecting the presence, absence and/or amount of an allergen in a sample is affected by the efficiency with which these proteins are isolated from the samples, in addition to the efficiency of antibodies used in the present invention.

In some embodiments, samples are processed and allergen proteins are extracted to ensure a fast, reliable and sensitive detection assay. The sample size and weight, extraction solution and extraction procedures may be optimized for an effective and non-destructive reaction. For example, the consumable sample in US patent publication NO. US2015/0011020 to Sundvor et al, may be measured and pre-processed before the consumable sample is delivered to the first chamber 110 through the consumable reception opening 112 (FIG. 2).

In some embodiments, a means for weighing may be provided when collecting a portion of a test sample for the detection assay. The collected portion may be weighed to ensure an adequate amount of the test sample being collected. The measured portion of the test sample may range from about 0.1 g to about 10 g, or about 1 g to about 5 g, or about 5 g to about 10 g. The sample needed for an accurate detection test may vary for different food matrices. In one aspect, a portion of about 5 g of a sample is collected and will be processed for a detection assay. In other aspects, a sample approximately 0.5 g of food may be enough to allow detection of traces of allergens. The means for weighing a sized sample may be a spring, a scale, a weighing tension module, or the equivalent thereof. Such weighed test sample may be delivered to the first chamber 110 through the consumable reception opening 112 (FIG. 2).

In some embodiments, a test sample may be pre-processed prior to being transmitted to a first sample receiving chamber 110 of a test container 105 in the detection system 100, or any similar processing chamber in a detection device. For example, the collected sample may be cut into small pieces, or be crushed into powder, or be smashed into small particles, or be heated up to a liquid state. Such pre-processes will facilitate further homogenization in the sample receiving chamber 110, or any similar processing chamber of a detection device and assist in sample digestion and efficient allergen protein extraction.

In some embodiments, a universal protein extraction buffer may be used to retrieve enough target proteins (e.g. allergens) (minimum 2 mg/ml total protein) for analysis from any food matrix. In some embodiments, the formulation of the universal protein extraction buffer can extract the protein at room temperature and in minimal time (e.g., less than 5 minutes). In some aspects, allergen proteins may be extracted in less than about 5 minutes, less than about 4 minutes, less than about 3 minutes, or less than about 2 minutes, or less than about 1 minute, or less than about 45 seconds, or less than about 30 seconds. The buffer may need to be incorporated with an extraction protocol that will include food sampling, homogenization and filtration. The extraction protocol may be implemented in a way that is efficient and repeatable over time and in different food matrices. This universal formulation will be clinically relevant as to try to minimally affect the food tested. This optimized protein extraction process will provide a fast, accurate and universal protocol that allows detection of an allergen in any food matrix.

This universal extraction buffer can maximize protein extraction and allergen retrieval. The universal extraction buffer will be applicable to any allergen and to all foods (e.g. pre-processed or post-processed food products). Additionally, the universal extraction buffer can improve antibody binding affinity, minimize non-specific binding and increase signal to noise ratio.

In other embodiments, the extraction buffer may be specialized for a particular type of sample, for example, a cooked meat.

In some embodiments, the extraction buffer of the present invention may be used as the processing reagent in the second chamber 130 of the test container 105 (FIG. 2)

Additionally, allergen protein extraction procedures may be optimized to obtain an adequate amount of proteins to achieve reliable detection results. The extraction processes may vary dependent on the types of food samples tested.

In one example, a measured test sample is processed in the first chamber 110 of the disposable test container 105, as disclosed in the detection system 100 in US Patent Publication NO.: 2015/0011020, to Sundvor et al (FIGS. 1 and 2), or any similar sample processing chamber of an allergen detection device, through homogenization. A homogenizer assembled in such a chamber will process the measured and/or pre-processed sample to facilitate the generation of a homogenized sample. The processed sample may be delivered to the second chamber 130 through the second opening 114 in the test container 105 (FIG. 2). The homogenized sample is mixed with a process reagent, which may be a universal extraction buffer or a specialized extraction buffer for a particular antigen, and is digested in the buffer to produce a dispersion. The homogenizer may be the driving element 120 of US Patent publication NO. 2015/0011020 to Sundvor et al. (FIG. 2). Any mechanisms that can break samples such as cutting, grinding, blending and filtration may be used, alone or in combination, to process a sample. In accordance with the present invention, the homogenizer may be provided with one or more bladders, grinders (e.g., the grinder 122 of FIG. 3), blenders or a set of protrusions arranged in a uniform distribution at one end (e.g., a set of protrusion 124 coupled to the shaft 123 of the grinder 122 shown in FIG. 3), and/or the combination thereof. In some aspects, the homogenizer is assembled in a food processing chamber in a detection device and controlled by a motor integrated in the detection device (e.g., the first chamber 110 of the portable test container 105 in the detection device 100 U.S. Patent Publication NO. 2015/0011020 to Sundvor et al.). In some cases, homogenizers of the invention such as the driving element 120 (FIG. 2) may further be provided with a heating and/or cooling mechanism to control sample and/or device temperature during homogenization.

Allergen proteins and other proteins as well, are extracted from the homogenized sample. During the protein extraction, the homogenized sample is drived from the first chamber 110 to the second chamber 130. Samples may be circulated within the portable test container and detection device by any type of means, including, but not limited to by gravity, air pressure, by vacuum pressure, by filtration, by absorption, by diffusion, and by diaphragm pump. As used herein, a “diaphragm pump” refers to a pump that functions by positive displacement using a reciprocating membrane (e.g., plastic, rubber, or other flexible membrane material) covering a chamber with valves on either side to control flow. In some cases, samples are circulated between chambers of devices of the invention. In some cases, processed samples from a first device chamber are delivered to an analytic chamber by air pressure, vacuum pressure, filtration, or diaphragm pump.

In one example, the homogenized sample is drived from the first chamber 110 to the second chamber 130 by the plunger 128 (FIG. 3). As described by Sundvor et al (US2015/0011020), the plunger 128 may drive homogenized sample portions into the second chamber 130 after and/or during the process by the grinder 122. Additionally, the plunger 128 may function to transition a diaphragm 160 between configurations to facilitate transferring the homogenized sample to the second chamber 130 (FIGS. 2 and 3).

As discussed previously, the homogenized sample may be further processed to extract allergen proteins before being detected for the absence, presence and/or amount of allergen in the sample. In some embodiments, the homogenized sample is delivered to the second chamber 130 through the sample reception opening 132 and mixed with a process reagent packaged in the second chamber 130, i.e., a universal extraction buffer or a specified buffer for a particular sample, using the mixing element 134 in the chamber, to produce a dispersion (FIGS. 2 and 3). In other embodiments, the extraction buffer may be provided during the process of a detection assay by a fluid delivery module, and mixed with the homogenized sample in the second chamber 130. The fluid delivery module may be coupled to the second chamber 130 or any other suitable portion of the test container 105 (FIG. 2). In one example, the extraction buffer may be delivered from a module that is integrated with one portion of the homogenizer such as the driving element 120 in FIG. 2. The extraction buffer is delivered to the first chamber 110, wherein the test sample is homogenized and proteins are extracted, simultaneously. The protein extraction is drived to the second chamber 130 wherein it is further delivered to an analytic chamber for allergen detection.

In some embodiments, the protein extraction may take place at ambient temperature or in a heated environment if needed. For Instance, it is hard to detect soybean proteins in fermented soybean products. A heating process may be used in combination with the extraction method to inhibit soybean protein degradation by microbial proteolytic enzymes in fermented soybean products. Such extraction method enables the sensitive detection of soybean proteins present at low concentrations in fermented soybean products. In other embodiments, a cooling system may be provided during homogenization and protein extraction. Some antibodies may recognize only an intact structure of an allergen protein. Low temperature will be beneficial to maintain the natural and intact structure of allergen proteins.

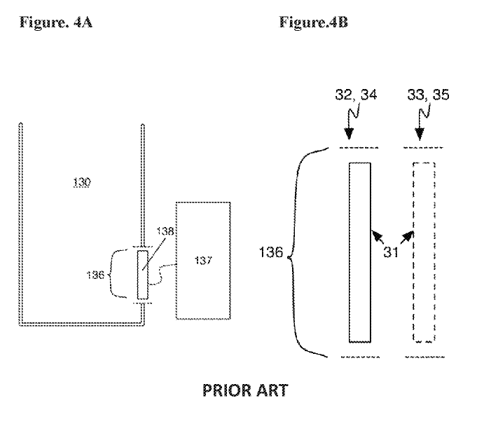

In some embodiments, the extracted protein solution may be filtered prior to the detection assay. In one example, the filter assembly may be provided in the second chamber 130 of the test container 105 by Sundvor et al (US2015/0011020). The filter assembly may be a membrane filter coupled to the outlet port 136 of the second chamber 130. The filtered sample solution may be delivered to an analytical chamber for allergen detection. In some embodiments, the analytical chamber is the analysis chamber 140 of the test container 105 by Sundvor et al (US2015/0011020). As discussed above, the sample solution may be circulated from the second chamber 130 to the analysis chamber 140 by any controlled means (e.g., gravity, air pressure, pumping, etc.). The outlet port 136 or other suitable modules may be used to control the flow of the sample solution to allow an adequate volume of the sample solution to be transmitted to the analysis chamber 140 (FIGS. 2 and 3) wherein the adequate sample solution is enough to provide a detectable signal. One example of the module that controls the flow may be an actuation system 137 which is coupled to the outlet 136 through the valve 138, as taught by Sundvor et al (US2015/0011020) (FIGS. 4A and 4B). The valve may be configured to transmit from one first configuration to the second configuration to allow a controlled flow of the sample solution passing through the outlet 136 to the analysis chamber 140.

In order to provide an accurate and reliable detection result in an allergen detection assay, total proteins extracted from a test sample are measured. The total proteins extracted from a test sample may be determined using any protein assays known to a skilled artisan in the field, e.g., bicinchoninic acid assay (BCA). In some aspects, a protein indication molecule (e.g., Pyrogalbl Red Molybdate, PRM) is used to determine the total protein. Any signal detected from the detection agent-allergen interaction will be normalized by the total protein measurement. In one example, the sample solution in the second chamber 140 of the test container 105 in US patent publication NO. 2015/0011020 to Sundvor et al. may be delivered to one defined part of the analysis chamber 140, or to a separated chamber in the test container 105, or any other portion of the test container 105, which is configured for measuring the total protein extracted. In some cases, the outlet port 136 and/or the actuation system 137 including the valve 138 may be used to control the flow and allow an adequate portion of the sample solution used for measurement of the total protein extraction (FIGS. 4A and 4B).

In other embodiments, a portion of the sample solution in the second chamber 140 of the test container 105 may be drived to yet another separate chamber for measuring the non-specific background, e.g., non-specific antibody binding to other proteins in the sample.

In the analytical chamber of the detection device, the processed sample solution react with one or more detection agents such as antibodies specific to allergens discussed previously for detecting the absence, presence and amount of an allergen of interest in the sample. In some embodiments, detection agents may be provided to the detection substrate 150 within the analysis chamber 140 of US patent publication NO. 2015/0011020 (FIGS. 1, 2, 3 and 5). The detection substrate 150 containing one or more antibodies may be positioned proximally to the outlet port 136 as shown in FIGS. 1, 2 and 3, and contact with the sample solution. The reaction between detection agents on the detection substrate 150, e.g., on the active region 151 of the substrate, and the sample solution indicates the absence, presence and/or amount of the allergen in the test sample.

In some embodiments, allergen detection assays and methods of the present invention provide a calibration standard (i.e. calibration curves) for a particular allergen and an antibody used. The calibration standard of a particular allergen protein may be generated from a raw or processed material that contains such allergen, or a purified allergen.

In some embodiments, allergen detection assays and methods of the present invention can detect a lower concentration of allergen in a food sample. The concentration of an allergen may be as low as 0.0001 ppm. In some aspects, the concentration or mass of allergen that can be detected may range from 0.001 ppm to 5 ppm, or from 0.001 ppm to 0.1 ppm, or from 0.1 ppm to 3 ppm, or from 1 ppm to 5 ppm, or from 5 ppm to 10 ppm. In some aspects, the concentration or mass of allergen in a food sample that can be detected may be 0.001 ppm, 0.002 ppm, 0.003 ppm, 0.004 ppm, 0.005 ppm, 0.006 ppm, 0.007 ppm, 0.008 ppm, 0.009 ppm, 0.01 ppm, 0.02 ppm, 0.03 ppm, 0.04 ppm, 0.05 ppm, 0.06 ppm, 0.07 ppm, 0.08 ppm, 0.09 ppm, 0.1 ppm, 0.2 ppm, 0.3 ppm, 0.4 ppm, 0.5 ppm, 0.6 ppm, 0.7 ppm, 0.8 ppm, 0.9 ppm, 1.0 ppm, 1.5 ppm, 2 ppm, 2.5 ppm, 3 ppm, 3.5 ppm, 4 ppm, 4.5 ppm, 5 ppm or 10 ppm.

In some embodiments, allergen detection assays and methods of the present invention may complete the implementation in less than 1 hour. In some aspects, the assay time may be from about 60 minutes to about 30 minutes, or about 40 minutes to about 10 minutes, or from about 20 minutes to about 5 minutes. In other embodiments, the assay time may be less than 5 minutes, such as from about 1 minute to about 5 minutes, about 1 minute to about 3 minute, about 2 minutes to about 10 minutes, or about 5 minutes to about 10 minutes. In other aspects, the assay time may last less than about 1 minute, about 2 minutes, about 3 minutes, about 4 minutes, about 5 minutes, about 6 minutes, about 7 minutes, about 8 minutes, about 9 minutes, or about 10 minutes. In further other aspects, the assay time may last less than about 10 seconds, about 15 seconds, about 20 seconds, about 25 seconds, about 30 seconds, about 35 seconds, about 40 seconds, about 45 seconds, about 50 seconds, about 55 seconds, or about 60 seconds.

In some embodiments, the methods of the present invention may detect an allergen in samples containing less than 0.1% allergen. In some aspects, the allergen concentrations of less than 0.5%, or less than 1%, or less than 5%, or less than 10% may be determined.

In some embodiments, the detected signals from interaction between amino acid-based allergens in the test sample and detection agents may be analyzed and compared with the total protein measurement, background signal and/or standard curves of the allergen concentration to provide the absolute amount of allergen proteins presented in the test sample.

In some embodiments, methods and assays of the present invention are accurate, timely and sensitive in detecting an allergen in a sample. In particular, such methods may reduce the detection time for a portable detection device used by an individual who looks for an immediate result on the allergen content of a food source.

In some embodiments, detection methods of the present invention are repeatable and sensitive.

In accordance with the present invention, methods and systems used to detect and display the antibody and allergen interaction may be used to display the detection results.

In some aspects, the detection may be based on electrochemical signals. In such systems, the interaction between an antibody and an allergen of interest in an immunoassay may be electrochemically detected through stripping analysis of enzymatically (using alkaline phosphatase) deposited electrochemical indicator, which detect mass and charge transfer during antibody and target interaction. According to this method, antibodies are loaded to an electrode and an electrochemical indicator is bound to a target of interest. Electrochemical indicators may include, but are not limited to, methylene blue (MB), and gold nanoparticles.

In some embodiments, signals detected in an immunological assay may be colorimetric changes. Antibodies can be labeled with an enzyme such as HRP. During the immunological assay, a substrate of the enzyme conjugates may be added to the reaction area and a colorimetric change is indicative of the presence, absence or amount of the allergen being tested.

In some embodiments, allergen detection assays may depend on fluorescence emission signal from fluorescence resonance energy transfer (FRET). Antibodies are labeled with one or more fluorophores. The specific interaction between an antibody and an allergen of interest will generate a specific fluorescent signal.

In some embodiments, the antibody-allergen interaction in the present detection methods may be detected using a fiber-optic chemifluorescent immunoassay in which the amount of allergen is measured on the basis of the intensity of fluorescence amplified by an enzymatic reaction between the labeled enzyme by a detection antibody and a fluorescent substrate.

In certain embodiments, an optical assembly may be used to detect the interaction between an antibody and a target allergen. The optical assembly may comprise a light emitting diode (LED) that provides light of an excitation wavelength appropriate to excite the fluorophore conjugated on the antibody. The fluorescence emitted from the fluorophores of the antibodies may be filtered and only the wavelength(s) of interest is transmitted. A means then may be used to process and convert the fluorescence signals to useful readouts (i.e. digital signals).

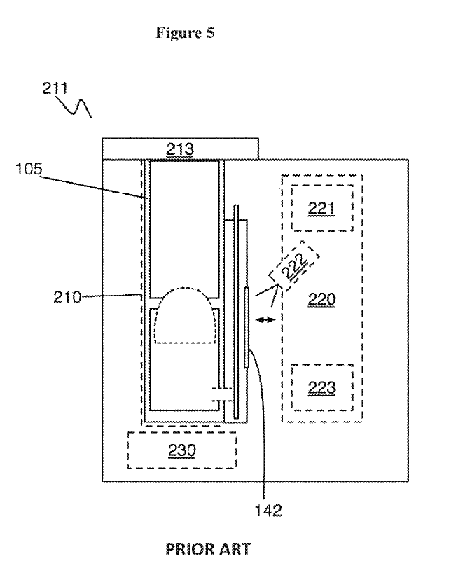

In one example, the detection and signal analysis may be implemented using the optical sensing subsystem 220 of the analysis device 205 in US patent publication NO. 2015/0011020 to Sundvor et al (FIG. 5). The optical sensing subsystem 220 may comprise an illumination module 222 which can illuminate the substrate 150 which is loaded with detection agents such as antibodies and has been reacted with the sample solution. A photodiode system 223 may be included in the optical system 220 to detect the absorption and emission of light. A camera module 221 and other suitable imaging techniques may be included to provide the data from the interaction between antibodies and allergens from the sample (FIG. 5).

In some embodiments, signals generated from the interaction of antibody and allergen may be detected in a separate analytic chamber of a disposable test container or the like in a detection device. The detection may be through a detection window adjacent to the analytic chamber. In one example, the detection result may be viewed through a detection window 142 included in the analysis chamber 140 in US patent publication NO. 2015/0011020 to Sundvor et al.

The detection result from the present assay may be displayed in a platform that a user can easily read such as a display window. In one embodiment, it may be a platform application in a cellphone (Coskun et al., A personalized food allergen testing platform on a cellphone, Lab Chip., 2013, 13(4), 636-640; the content of which is incorporated herein by reference in its entirety.)

In some examples, methods and assays of the present invention may be used in the detection system disclosed by Sundvor et al in US Patent publication NO. US2014/0295406 and PCT publication NO. 2014/160861; the content of each of which is incorporated by reference in their entirety.

In other examples, methods and assays of the present invention may be used in the self-testing allergen test kit by Choo and Makower, et al. in PCT publication NO. 2012/078455; the content of which is incorporated by reference in its entirety.

In some embodiments, methods and assays of the present invention may be used in any allergen detection devices and systems. Some non-limiting examples include lateral flow devices (LFD), microfluidic chips (U.S. Pat. No. 8,617,903), detection device of Sundvor et al., (US Patent Publication NOs.: 2014/0295406 and 2015/0011020), and portable detection devices/systems described in the commonly owned U.S. patent application No. 62/133,632 filed on Mar. 16, 2015 and the cartridge as described in the commonly owned PCT patent application NO.: PCT/US14/62656 filed on Oct. 28, 2014, each of which is incorporated herein by reference in its entirety.

### Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

About: As used herein, the term “about” when referring to a measurable value such as an amount of weight, time, dose, etc. is meant to encompass variations of ±20% or ±10%, more preferably ±5%, even more preferably ±1%, and still more preferably ±0.1% from the specified amount, as such variations are appropriate to perform the disclosed method.

Activity: As used herein, the term “activity” refers to the condition in which things are happening or being done. Compositions of the invention may have activity and this activity may involve the binding to a target molecule.

Allergen: as used herein, the term “allergen” means a compound, substance or composition that causes, elicits or triggers and immune reaction in a subject. As such, allergens that cause the production of antibodies are typically referred to as antigens. Allergens may be any chemical compounds, including, but not limited to organic compounds (e.g., amino acid-based compounds, such as peptides and proteins).

Allergen detection agent: As used herein, the terms “allergen detection agent” or “detection agent” refers to any molecule which is capable of, or does, interact with and/or bind to one or more allergens in a way that allows detection of such allergens in a sample. Detection agents of the present invention may include, but are not limited to, antibodies that can specifically bind to an allergen.

Biomolecule: As used herein, the term “biomolecule” is any natural molecule which is amino acid-based, nucleic acid-based, carbohydrate-based or lipid-based, and the like.

Detection: As used herein, the term “detection” means an extraction of a particular target protein from a mixture of many non-target proteins, indicating the absence, presence, and/or amount of a target protein from a mixture of many non-target proteins.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance, immunological detection and the like. Detectable labels may include radioisotopes, fluorophores, chromophores, enzymes, dyes, metal ions, ligands, biotin, avidin, streptavidin and haptens, quantum dots, polyhistidine tags, myc tags, flag tags, human influenza hemagglutinin (HA) tags and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Epitope: As used herein, an “epitope” refers to a surface or region on a molecule that is capable of interacting with a detection agent (e.g., an antibody). Epitopes may include, but are not limited to, one or more feature, region, domain, chemical group, functional group, or moiety. As such, epitopes may also include, but are not limited to, one or more atom, group of atoms, atomic structure, molecular structure, cyclic structure, hydrophobic structure, hydrophilic structure, sugar, lipid, amino acid, peptide, glycopeptide, or nucleic acid molecule. In some embodiments, when referring to a peptide or protein, an epitope may comprise a linear stretch of amino acids or a three dimensional structure formed by folded amino acid chains.

Including: As used herein, the term “including” refers to “including but not limited to”. “Including” and “including but not limited to” are used interchangeably.

Interaction: As used herein, the term “interaction” refers to a kind of action that occurs as two or more molecules have effect upon one another. In the context of the present invention, an interaction between a detection agent such as an antibody and a target affects the structure of the detection agent and such effect will generate energetic changes that can be visualized.

Pathogen: As used herein, the term “pathogen” means any disease-producing agent (especially a virus or bacterium or other microorganism).

ppm: As used herein, the term “ppm” is an abbreviation of parts per million. ppm is a value that represents the part of a whole number in units of 1/1000000. ppm is dimensionless quantity, a ratio of 2 quantities of the same unit. For example: mg/kg. One ppm is equal to 1/1000000 of the whole: 1 ppm= 1/1000000=0.000001=1×10⁻⁶. ppm herein is used to measure chemical (protein) concentration, usually in a solution. Solute concentration of 1 ppm is solute concentration of 1/1000000 of the solution. The concentration C in ppm is calculated from the solute mass mₛₒₗᵤₜₑ in milligrams and the solution mass mₛₒₗᵤₜᵢₒₙ in milligrams: (C₍ₚₚₘ₎=1000000 mₛₒₗᵤₜₑ/(mₛₒₗᵤₜᵢₒₙ+mₛₒₗᵤₜₑ).

Sample: As used herein, the term “sample” refers to any composition that might contain a target of interest to be analyzed including, but not limited to, biological samples obtained from subjects (including humans and animals as detailed below), samples obtained from the environment for example soil samples, water samples, agriculture samples (including plant and crop samples), or food samples. Food samples may be obtained from fresh food, processed/cooked food or frozen food.

Sensitivity: As used herein, the term “sensitivity” means the ability of a detection agent (e.g., an antibody) to bind to a target molecule.

Specifically bind(s): As used herein, the term: specifically bind(s)” means that a detection agent (e.g., antibody) reacts or associates more frequently, more rapidly, with greater duration and/or with greater affinity with a particular target such as an allergen protein than it does with alternative targets. For example, an antibody that specifically binds to an allergen protein binds that protein or a fragment thereof with greater affinity, avidity, more readily, and/or with greater duration than it binds to unrelated protein and/or the fragments thereof. It is also understood by an artisan by this definition, for example, a detection agent (e.g., antibody) that specifically binds to a first target may or may not specifically bind to a second target. As such, “specific binding” does not necessarily require exclusive binding or non-detectable binding of another molecule, this is encompassed by the term “selective binding”. Generally, but not necessarily, reference to binding means specific binding. The specificity of binding is defined in terms of the comparative dissociation constants (Kd) of the antibody for target as compared to the dissociation constant with respect to the antibody and other materials in the environment or unrelated molecules in general. Typically, the Kd for the antibody with respect to the target will be 2-fold, 5-fold, or 10-fold less than the Kd with respect to the target and the unrelated material or accompanying material in the environment. Even more preferably, the Kd will be 25-fold, 50-fold, 75-fold, 100-fold, 150 fold or 200-fold less.

Target: as used herein, the term “target” and “target molecule” refers to a molecule which may be found in a tested sample and which is capable of binding to a detection agent such as an aptamer or an antibody. Targets may also refer to target allergens.

Universal buffer: As used herein, the term “universal buffer” refers to a buffer that may be used for a variety of samples.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

### Examples

### Example 1: Food Sampling

A sampling means is tested and optimized to ensure reliable and reproducible food sampling, with high accuracy between different food matrices. A number of different food samples such as baked goods (e.g., cake and bread), diary (e.g., cheddar cheese, baby formula and peanut butter cup ice cream), snacks (e.g., Peanut butter filled pretzels and Twinkies (TWINKIE®)), chocolate, fish and seafood (e.g., clam chowder and canned shrimp), and meats (e.g., pork sausages and chicken nuggets) are tested. The sampling process begins with the insertion of a probe into a target food sample and captures a portion of the target sample.

Optionally a weighing mechanism is attached to the sampling means and the picked sample is weighed to ensure a specific amount of sample collected for a reliable and accurate detection test. The sampling mechanisms vary dependent on food samples to be monitored. A measured portion of the sample is subject to sample digestion and protein extraction. A table is created, comparing the total protein, specific allergen recovery in various weights and sizes, and by different processing mechanisms, and in different optimized extraction buffer as well (as discussed in the following examples). Sample homogenization is carried out through grinding, blending, cutting, crushing, nibbling, smashing, vibrating and/or a combination thereof. All of these optimizations are done in the context of time consideration and the goal is to reduce the process time to less than 5 minutes.

### Example 2: Allergen Protein Extraction and Extraction Buffer

The first step to develop a sensitive detection assay is to optimize and unify the protein extraction process with a fast, accurate and universal protocol that allows detection of allergen in any food matrix. Various extraction buffer protocols that will include reduction, and blocking agents, detergents and surfactants, are tested and compared.

The chemicals that are suitable for universal sampling are tested and optimized, including Beta-Mercaptoethanol 0.5-5%, Dithiothreitol (DTT) 0.2-2.5%, Deoxycholate sodium, Sodium Dodecyl Sulfate (SDS), NP-40/Triton100/Tween20, MgCl₂/KCl and/or Gelatin/BSA/Skim milK. Buffers such as Phosphate based buffer and Tris buffer are tested and optimized for different concentrations of agents, pH values of buffers, or with addition or substitute of one or more agents. The proteins extracted in optimized buffers are measured and compared. A comparison of these tested buffers to known extraction buffers used in the food allergen field today primarily for ELISA testing, by testing at least three different ELISA assay for each allergen protein is performed. The optimal extraction buffer may be selected through a structured process that will test and compare up to 20 different food matrices known to be tested in the optimization of ELISA assays (e.g., baked goods, sausages, soups, ice cream, etc.), as well as more challenging food matrices (e.g., salad dressing, soy sauce and chocolate).

As an example of a selection process, about 0.5 mg food sample is spiked with known amounts of allergens before and after processing (baking, boiling, frying etc.). Total protein extraction as well as specific allergen retrieval is tested and recorded to compare efficiency of different extraction buffers. The readout will be total protein extraction and relative recovery of specific allergen (% of amount spiked). The deliverable will be a table comparing the total protein, specific allergen recovery (such as egg, wheat, peanut, fish, crustacean, milk, cashew, soy) before and after processing in different optimized buffers compared to 3 different extraction buffers from commercial ELISA assays.

Allergen proteins are extracted from a food sample by a mechanism that is suitable for the sample type such as blending, cutting, blinding or the like. An extraction buffer is used to process the sample. We then further test the sample size, methods for preparing protein samples and physical programs and their influence on allergen recovery and antibody binding affinity to allergen.

### Example 3: Total Protein Measurement

Total proteins extracted from a test sample vary and could influence the detection test. In this study, total protein measurement is tested using Pyrogallol Red-molybdate (PRM) protein dye-binding assays. PRM is first made in a solution containing 0.156 mM pyrogallol red, 0.209 mM sodium molybdate and 50 mM Tris-HCl. A test plate is prepared by adding 20 μl/well PRM solution and the plate is dry overnight. After processing the test food matrices, processed sample solution (400 μl) is added to each well and the protein absorbance is read immediately at 600 nm.

### Example 4: Examination with Purified Allergens

This example describes one procedure for identifying and quantifying the presence of an allergen protein in a food sample according to the optimized procedures described in the present application.

A food sample of interest is prepared and serial dilutions of the sample are prepared. In one experiment, about 100 ul of sample dilution is loaded to each well of a 96-well plate. A serial dilution of an antibody that specifically recognizes an allergen protein presented in the test sample is prepared. The antibodies are labeled with a fluorophore. In another experiment, a secondary antibody that specifically binds to the primary antibody is used and labeled with a fluorophore. The antibody dilutions are added to each well of the 96-well plate and incubated with the sample solution and the signaling is read and measured for the concentration of the allergen in the test sample.

1. A method for detecting an allergen in a test sample comprising:
a). obtaining a test sample,
b). receiving the test sample in a first chamber of a test container in a detection device through a reception port of the first chamber configured to receive the test sample,
c). processing the test sample in the first chamber by homogenization,
d). delivering the homogenized sample to a second chamber of the test container through a second port of the first chamber, wherein said second port is configured to deliver the homogenized sample in the first chamber to the second chamber, and wherein the second chamber is configured to receive the homogenized sample from the second port of the first chamber,
e). processing the homogenized sample in an extraction buffer in the second chamber of the test container;
f). contacting the processed sample with one or more detection agents, wherein said one or more detection agents specifically bind to said allergen, and
g). observing a detection signal.

2. The method of claim 1 further comprising pre-processing the test sample in step (a) prior to receiving the test sample in the first chamber of the test container in the detection device, wherein the test sample is pre-processed by cutting into small pieces, crushing into powder, smashing into small particles, heating, or a combination thereof.

3. (canceled)

4. The method of claim 1, wherein the extraction buffer is optimized to achieve a maximal extraction of the allergen from the test sample.

5. The method of claim 1, wherein the step (e) further comprises providing the one or more detection agents to the analytic chamber within the test container.

6. The method of claim 1, wherein the one or more detection agents are confined to a surface in the analytic chamber within the test container.

7. The method of claim 6, wherein said surface is selected from the group consisting of a test strip, a membrane on a test strip, a plastic surface, and a glass surface.

8. The method of claim 1, wherein the one or more detection agents comprise one or more antibodies which specifically bind to the allergen.

9. The method of claim 8, wherein the antibody is selected from a monoclonal antibody, a polyclonal antibody, an antibody fragment, and an antibody variant, and wherein said antibody comprises a detectable label selected from the group consisting of a fluorescent label, a luminescent label, an enzymatic label, and a radioactive label.

10. (canceled)

11. (canceled)

12. The method of claim 8, wherein the binding of said one or more antibodies to the allergen is detected with a secondary antibody, wherein said secondary antibody comprises a detectable label selected from the group consisting of a fluorescent label, a luminescent label, an enzymatic label, and a radioactive label.

13. (canceled)

14. The method of claim 1, wherein the processed sample from the first chamber is delivered to the analytic chamber by a means selected from air pressure, vacuum pressure, filtration, and diaphragm pump.

15. The method of claim 1, wherein the first chamber comprises a homogenizer configured for homogenizing the test sample and is controlled by a motor within the detection device.

16. The method of claim 15, wherein the homogenizer is provided with a heating and/or cooling mechanism.

17. The method of claim 1, wherein the extraction buffer is stored in the first chamber and contacted with the test sample during the homogenization in step (c).

18. The method of claim 1, wherein the step (c) further comprises providing the extraction buffer to the first chamber during the homogenization.

19. The method of claim 1, wherein said allergen is selected from the group consisting of a food allergen, an allergen from the environment, and a medical allergen.

20. The method of claim 19, wherein said allergen is a food allergen which comprises an allergenic protein associated with said food allergen.

21. The method of claim 20, wherein said food allergen is peanut which comprises an allergenic protein selected from the group consisting of Ara h 1, Ara h 2, Ara h 3, Ara h 4, Ara h 5, Ara h 6, Ara h 7, Ara h 8, Ara h 9, Ara h 10, Ara h 11, Ara h 12, and Ara h 13.

22. The method of claim 1, wherein said method is completed in less than 5 minutes.

23. The method of claim 1, wherein at least one standard sample is also analyzed according to the same method and wherein the detection signal obtained from said processed sample is compared to that obtained from said at least one standard sample to obtain the level of said allergen in said processed sample.

24. The method of claim 23, wherein the allergen is present in the test sample at a concentration of less than 0.1%.
